SEQUENCE LISTING (32 sequences)

SEQ ID NO: 1 - RNA, 56 nt, Artificial Sequence
  Synthetic oligonucleotide
ggcugguccgaugguaguggguuaucagaacuuauuaacauuagugucacuaaagu

SEQ ID NO: 2 - RNA, 24 nt, Artificial Sequence
  Synthetic oligonucleotide
  misc_feature (1)..(1): LNA
  misc_feature (3)..(3): LNA
  misc_feature (5)..(5): LNA
  misc_feature (20)..(20): LNA
  misc_feature (22)..(22): LNA
  misc_feature (24)..(24): LNA
cguccgaugguaguggguuaucag

SEQ ID NO: 3 - DNA, 22 bp, Artificial Sequence
  Synthetic oligonucleotide
ggtccgatggtagtgggttatc

SEQ ID NO: 4 - DNA, 22 bp, Artificial Sequence
  Synthetic oligonucleotide
ggtccgaaggtagtgagttatc

SEQ ID NO: 5 - DNA, 24 bp, Artificial Sequence
  Synthetic oligonucleotide
ggtccgatggtagtgggttatcag

SEQ ID NO: 6 - RNA, 24 nt, Artificial Sequence
  Synthetic oligonucleotide
gguccgaugguaguggguuaucag

SEQ ID NO: 7 - RNA, 54 nt, Artificial Sequence
  Synthetic oligonucleotide
cugguccgaugguaguggguuaucagaacuuauuaacauuagugucacuaaagu

SEQ ID NO: 8 - RNA, 54 nt, Artificial Sequence
  Synthetic oligonucleotide
cugguccgaugguaguggguuaucagaacuuauuaacauuagugucacuaaaga

SEQ ID NO: 9 - RNA, 54 nt, Artificial Sequence; identical to SEQ ID NO: 8
  Synthetic oligonucleotide
  misc_feature (1)..(1): LNA
  misc_feature (2)..(2): LNA
  misc_feature (3)..(3): LNA
  misc_feature (52)..(52): LNA
  misc_feature (53)..(53): LNA
  misc_feature (54)..(54): LNA

SEQ ID NO: 10 - RNA, 54 nt, Artificial Sequence; identical to SEQ ID NO: 8
  Synthetic oligonucleotide
  misc_feature (1)..(1): LNA
  misc_feature (3)..(3): LNA
  misc_feature (5)..(5): LNA
  misc_feature (50)..(50): LNA
  misc_feature (52)..(52): LNA
  misc_feature (54)..(54): LNA

SEQ ID NO: 11 - RNA, 24 nt, Artificial Sequence
  Synthetic oligonucleotide
gguccgaugguaguggguuaucag

SEQ ID NO: 12 - RNA, 24 nt, Artificial Sequence
  Synthetic oligonucleotide
cguccgaugguaguggguuaucag

SEQ ID NO: 13 - RNA, 24 nt, Artificial Sequence
  Synthetic oligonucleotide
  misc_feature (1)..(1): LNA
  misc_feature (2)..(2): LNA
  misc_feature (3)..(3): LNA
  misc_feature (22)..(22): LNA
  misc_feature (23)..(23): LNA
  misc_feature (24)..(24): LNA
cguccgaugguaguggguuaucag

SEQ ID NO: 14 - DNA, 21 bp, Artificial Sequence
  Fomivirsen
gcgtttgctcttcttcttgcg

SEQ ID NO: 15 - DNA, 20 bp, Artificial Sequence
  Mipomersen
gccucagtctgcttcgcacc

SEQ ID NO: 16 - DNA, 18 bp, Artificial Sequence
  Nusinersen
ucacuuucauaaugcugg

SEQ ID NO: 17 - DNA, 30 bp, Artificial Sequence
  Eteplirsen
ctccaacatcaaggaagatggcatttctag

SEQ ID NO: 18 - DNA, 20 bp, Artificial Sequence
  Inotersen
tcttggttacatgaaatccc

SEQ ID NO: 19 - DNA, 21 bp, Artificial Sequence
  Patisiran
guaaccaagaguauuccautt

SEQ ID NO: 20 - DNA, 25 bp, Artificial Sequence
  Golodirsen
gttgcctccggttctgaaggtgttc

SEQ ID NO: 21 - DNA, 21 bp, Artificial Sequence
  Givosiran
cagaaagagugucucaucuua

SEQ ID NO: 22 - DNA, 21 bp, Artificial Sequence
  Viltolarsen
cctccggttctgaaggtgttc

SEQ ID NO: 23 - DNA, 20 bp, Artificial Sequence
  Volanesorsen
agcttcttgtccagctttat

SEQ ID NO: 24 - DNA, 21 bp, Artificial Sequence
  Inclisiran
cuagaccugutuugcuuuugu

SEQ ID NO: 25 - DNA, 21 bp, Artificial Sequence
  Lumasiran
gacuuucauccuggaaauaua

SEQ ID NO: 26 - protein, 214 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKDDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 27 - protein, 222 aa, Bos taurus
MKFFIFTCLLAVALAKNTMEHVSSSEESIISQETYKQEKNMAINPSKENLCSTFCKEVVRNANEEEYSIGSSSEESAVVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMEPWIQPKTKVIPYVRYL

SEQ ID NO: 28 - protein, 259 aa, Bos taurus
MPLNTIYKQPQNQIIIHSAPPSLLVLYFGKKELRAMKVLILACLVALALARELEELNVPGEIVESLSSSEESITRINKKIEKFQSEEQQQTEDELQDKIHPFAQTQSLVYPFPGPIHNSLPQNIPPLTQTPVVVPPFLQPEVMGVSKVKEAMAPKHKEMPFPKYPVEPFTERQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 29 - protein, 216 aa, Bos taurus
MKVLILACLVALALARELEELNVPGEIVESLSSSEKIEKFQSEEQQQTEDELQDKIHPFAQTQSLVYPFPGPIHNSLPQNIPPLTQTPVVVPPFLQPEVMGVSKVKEAMAPKHKEMPFPKYPVEPFTERQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 30 - protein, 190 aa, Bos taurus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV

SEQ ID NO: 31 - RNA, 26 nt, Homo sapiens
ccccccacugcuaaauuugacugguu

SEQ ID NO: 32 - RNA, 26 nt, Homo sapiens
ccccccacugcuaaauuugacuggua

(19) United States
(12) Patent Application Publication
(10) Pub. No.: US 2024/0285668 A1
(43) Pub. Date: Aug. 29, 2024

(54) FORMULATIONS FOR ORAL DELIVERY OF NUCLEIC ACIDS

(71) Applicant: Cedars-Sinai Medical Center, Los Angeles, CA (US)

(72) Inventors: Eduardo Marbán, Santa Monica, CA (US); Ahmed G. Ibrahim, Los Angeles, CA (US)

(21) Appl. No.: 18/572,067

(22) PCT Filed: Jun. 30, 2022
(86) PCT No.: PCT/US2022/035870

§ 371 Date: Dec. 19, 2023

Related U.S. Application Data
(60) Provisional application No. 63/202,970, filed on Jul. 1, 2021.

(51) Int. Cl. A61K 31/7088; A61K 9/00; A61K 9/107; A61K 47/12; A61K 47/36; A61K 47/42; A61P 9/04; A61P 9/10; A61P 21/00; C12N 15/113

(52) U.S. Cl. CPC A61K 31/7088; A61K 9/0053; A61K 9/1075; A61K 47/12; A61K 47/36; A61K 47/42; A61P 9/04; A61P 9/10; A61P 21/00; C12N 15/113

(57) ABSTRACT

Provided herein are formulations configured for the oral delivery of a nucleic acid, such as a non-coding RNA. The formulations provided for herein comprise a plurality of cationic lipids used to encapsulate the nucleic acid within a micelle and a mixture of casein proteins and chitosan polymers used to coat the lipids, which form a coating on the micelle. The coated micelle lends acid-resistance to the formulation such that oral administration is possible with enhanced bioavailability of the nucleic acid to conditions associated with inflammation or fibrosis, such as hypertrophic myocardiopathy, heart failure with preserved ejection fraction, muscle disorders, such as muscular dystrophy, scleroderma and/or viral infection.

### CROSS REFERENCE TO RELATED APPLICATIONS

The present application claims priority to U.S. Provisional Application No. 63/202,970, filed Jul. 1, 2021, the entire contents of which is incorporated by reference herein.

### STATEMENT REGARDING FEDERALLY SPONSORED R&D

This invention was made with government support under Grant No. R01 HL124074, awarded to Dr. Eduardo Marban by the National Institutes of Health. The Government has certain rights in the invention.

### REFERENCE TO SEQUENCE LISTING

The present application is being filed along with a Sequence Listing in electronic format. The Sequence Listing is provided as a file entitled CSMC018WO2_ST25.txt created on Jun. 30, 2022, which is 16,471 bytes in size. The information in the electronic format of the Sequence Listing is incorporated herein by reference in its entirety.

### BACKGROUND

### Field

The present disclosure relates to formulations for oral delivery of nucleic acids, such as therapeutic RNA (e.g., non-coding or coding RNA). In several embodiments, the formulations enhance the oral bioavailability of such RNAs such that oral delivery, rather than delivery by, for example, injection allows for treatment of various diseases, in particular those marked by inflammation and/or fibrosis.

### SUMMARY

In several embodiments, there is provided for herein a formulation for oral delivery of a nucleic acid, comprising a nucleic acid, a cationic lipid, at least one casein protein, and a chitosan.

In several embodiments, the nucleic acid of the formulation comprises a ribonucleic acid (RNA), wherein the RNA is present in an amount ranging between 0.0001 and about 0.01% of the formulation by weight per volume. In several embodiments, the at least one casein protein comprises at least an α-s1 casein subunit that is present in an amount ranging between about 0.25 and about 7% of the formulation by weight per volume, the chitosan is present in an amount ranging between about 0.0001 and 4% of the formulation by weight per volume.

In several embodiments, there is provided a formulation for oral delivery of a nucleic acid, comprising a plurality of artificial lipid micelles, a plurality of nucleic acids, wherein a portion of the nucleic acids are encapsulated within the artificial lipid micelles, and a coating on the artificial lipid micelles, wherein the coating comprises a mixture of casein proteins and chitosan polymers. In several embodiments, the nucleic acid comprises a ribonucleic acid (RNA) and wherein the RNA is present in an amount ranging between about 0.00001 and about 0.05% of the formulation by weight per volume, the mixture of casein proteins and chitosan polymers comprises at least an α-s1 casein subunit that is present in an amount ranging between about 0.5 and about 5% of the formulation by weight per volume, and wherein the chitosan is present in an amount ranging between about 0.001 and about 1% of the formulation by weight per volume.

In several embodiments, the formulation further comprises an acid. In several embodiments, the acid is present in an amount ranging between about 0.001 and about 1% of the formulation by volume and the acid is selected from acetic acid, citric acid, phosphoric acid and citric acid. In one embodiment, the formulation further comprises acetic acid, wherein the acetic acid is present in an amount ranging between about 0.01 and about 1% of the formulation by weight per volume.

In several embodiments, the cationic lipid is present in an amount ranging from about 0.1 to about 5 microliters for each microgram of nucleic acid. In several embodiments, the nucleic acid comprises a non-coding RNA, a coding RNA (e.g., mRNA), or a combination thereof. In several embodiments, the chitosan is low molecular weight chitosan. In several embodiments, the low molecular weight chitosan ranges in mass from about 50 to about 190 kiloDaltons.

In several embodiments, the nucleic acid comprises a non-coding RNA or a coding RNA, wherein the RNA is present in an amount ranging from between about 0.001 and about 0.005% of the formulation by weight per volume, wherein the at least one casein protein comprises a mixture of an α-s1 casein subunit, an α-s2 casein subunit, a β casein subunit, and a κ casein subunit, wherein the casein subunits are present in an amount ranging between about 1 and 3% of the formulation by weight per volume, and wherein the chitosan is present in an amount ranging between about 0.01 and 0.1% of the formulation by weight per volume.

In several embodiments, the nucleic acid comprises a non-coding RNA or a coding RNA, wherein the RNA is present in an amount ranging from between about 0.0015 and about 0.004% of the formulation by weight per volume, wherein the mixture of casein subunits are present in an amount ranging between about 2 and 3% of the formulation by weight per volume, wherein the chitosan is present in an amount ranging between about 0.05 and 0.1% of the formulation by weight per volume, and wherein the cationic lipid is present in an amount ranging from about 1 to about 3 microliters for each microgram of nucleic acid.

In several embodiments, the nucleic acid comprises a non-coding RNA or a coding RNA, wherein the RNA is present in an amount ranging from between about 0.0015 and about 0.0035% of the formulation by weight per volume, wherein the mixture of casein subunits are present in an amount ranging between about 2.2 and 2.8% of the formulation by weight per volume, wherein the chitosan is present in an amount ranging between about 0.06 and 0.09% of the formulation by weight per volume, and wherein the cationic lipid is present in an amount ranging from about 1 to about 2 microliters for each microgram of nucleic acid.

In several embodiments, upon administration to a subject, the RNA reduces expression of one or more of IL1-B, IL-6, TGF beta, NLRP3, p21, and IL-4. In several embodiments, upon administration to a subject, the RNA reduces systolic blood pressure of the subject. In several embodiments, upon administration to a subject, the RNA reduces diastolic blood pressure of the subject. In several embodiments, upon administration to a subject, the RNA enhances muscular endurance, muscular resistance to fatigue, muscular strength and/or muscle contractility of at least one muscle of the subject. Depending on the embodiment the muscle may be skeletal muscle or cardiac muscle. In several embodiments, upon administration to a subject, the RNA reduces the expression of brain natriuretic peptide.

In several embodiments, upon administration to a subject, the RNA reduces diastolic mitral inflow velocity to mitral annular tissue velocity (E/e′). In several embodiments, upon administration to a subject, the RNA enhances glucose tolerance within the subject. In several embodiments, upon administration to a subject, the RNA reduces obesity and/or subcutaneous adipose tissue per unit body mass of the subject. In several embodiments, upon administration to a subject having had a myocardial infarction, the RNA reduces infarct size after the myocardial infarction. In several embodiments, upon administration to a subject having had a myocardial infarction, the RNA reduces circulating cardiac troponin I concentration after the myocardial infarction.

In several embodiments, the formulation alleviates one or more symptoms of a disease associated with increased inflammation and/or fibrosis. In several embodiments, the disease is selected from heart failure with preserved ejection fraction, myocardial infarction, muscular dystrophy, scleroderma, viral infection, and hypertrophic cardiomyopathy. In several embodiments, the formulation comprises a nucleic acid having at least 90% sequence identity to one or more of SEQ ID NO: 1-25, 31, 32. In several embodiments, the formulation comprises a nucleic acid that consists essentially of a sequence having at least 90% sequence identity to one or more of SEQ ID NO: 1-25, 31, 32. In several embodiments, the formulation comprises one or more casein proteins having at least 80% sequence identity to one or more of SEQ ID NOs. 26-30.

Also provided for herein is a method for treating a disease that is associated with inflammation and/or fibrosis, comprising administering to a subject having the disease that exhibits inflammation and/or fibrosis a therapeutically effective amount of a formulation disclosed herein. Additionally provided is the use of a formulation disclosed herein for the treatment of a disease associated with inflammation and/or fibrosis. Additionally provided is the use of a formulation disclosed herein for the manufacture of a medicament for the treatment of a disease associated with inflammation and/or fibrosis. In several embodiments, the disease comprises heart failure with preserved ejection fraction, myocardial infarction, muscular dystrophy, scleroderma, viral infection, and/or hypertrophic cardiomyopathy.

In several embodiments, there is provided a method for manufacturing a formulation for oral delivery of a nucleic acid, comprising encapsulating a nucleic acid in an artificial lipid micelle by contacting the nucleic acid with a solution comprising cationic lipids, thereby generating an artificial lipid micelle comprising the nucleic acid, coating the artificial lipid micelle comprising the nucleic acid with casein proteins by contacting the artificial lipid micelle comprising the nucleic acid with a solution comprising between 2 and 10% casein proteins, thereby generating a casein coated artificial lipid micelle comprising the nucleic acid, and exposing the casein coated artificial lipid micelle comprising the nucleic acid to a mixture of an acid and chitosan polymers, wherein the mixture of the acid and the chitosan polymers allows intercalation of the chitosan with the casein proteins and precipitation of casein-chitosan coated lipid micelles comprising the nucleic acid.

In several embodiments, the nucleic acid is contacted with the cationic lipids in a ratio of between about 0.5 to 2.0 μL of lipid solution for each microgram of nucleic acid. In several embodiments, the method further comprise adding a liquid media to the nucleic acid and cationic lipid solution to a final volume of about 100 μL. In several embodiments, the casein proteins are within a solution of 5% bovine casein solution and are added to the artificial lipid micelle comprising the nucleic acid at a volume ratio of 1:10. In several embodiments, the mixture of the acid and the chitosan polymers comprises an acetic acid solution of between about 0.05 and 2% and a chitosan solution of between about 0.1% and 2%.

In several embodiments, there is provided a method of treating a disease associated with inflammation and/or fibrosis, comprising administering to a subject an oral formulation comprising a nucleic acid, a cationic lipid, at least one casein protein, and a chitosan. In several embodiments, the oral formulation is given on a daily basis. In several embodiments, a single oral administration is effective to ameliorate or otherwise treat the disease associated with inflammation and/or fibrosis. In several embodiments, the nucleic acid comprises a ribonucleic acid (RNA) and wherein the RNA is present in an amount ranging between about 0.00001 and 0.01% of the formulation by weight per volume, wherein the at least one casein protein comprises at least an α-s1 casein subunit and is present in an amount ranging between about 0.5 and 5% of the formulation by weight per volume; and wherein the chitosan is present in an amount ranging between about 0.001 and 1% of the formulation by weight per volume.

Provided herein is an isolated nucleic acid comprising a nucleotide sequence of CGUCCGAUGGUAGUGGGUUAUCAG (SEQ ID NO: 12), wherein the nucleic acid is RNA, wherein the nucleic acid is at most 30 nt long. Also provided is an isolated nucleic acid comprising a nucleotide sequence at least 95% identical to CGUCCGAUGGUAGUGGGUUAUCAG (SEQ ID NO: 12), wherein the nucleic acid is RNA, wherein the nucleic acid is at most 30 nt long. Optionally, the nucleic acid comprises at least one chemically-modified nucleotide. In some embodiments, the nucleic acid comprises between 1-10 chemically-modified nucleotides. In some embodiments, the nucleic acid comprises at least one chemically-modified nucleotide within positions 1-12 and/or at least one chemically-modified nucleotide within positions 13-24 of the nucleotide sequence. In some embodiments, the chemically-modified nucleotide comprises a backbone modification. In some embodiments, the backbone modification comprises a backbone sugar modification. In some embodiments, the nucleic acid comprises the chemically-modified nucleotide at one or more of positions 1, 3, 5, 20, 22 and 24 of the nucleotide sequence. In some embodiments, the chemically-modified nucleotide is a locked nucleic acid (LNA). In some embodiments, the nucleotide sequence comprises the LNA at positions 1, 3, 5, 20, 22 and 24 of the nucleotide sequence. In some embodiments, the nucleic acid is 24 nucleotides long.

Provided herein is an isolated nucleic acid comprising a nucleotide sequence at least 95% identical to CGUCCGAUGGUAGUGGGUUAUCAG (SEQ ID NO: 12), wherein the nucleic acid is RNA. Optionally, the nucleotide sequence is CGUCCGAUGGUAGUGGGUUAUCAG (SEQ ID NO: 12). In some embodiments, the nucleic acid comprises at least one chemically-modified nucleotide. In some embodiments, the nucleic acid comprises between 1-10 chemically-modified nucleotides. In some embodiments, the nucleic acid comprises at least one chemically-modified nucleotide within positions 1-12 and/or at least one chemically-modified nucleotide within positions 13-24 of the nucleotide sequence. In some embodiments, the chemically-modified nucleotide comprises a backbone modification. In some embodiments, the backbone modification is a backbone sugar modification. In some embodiments, the nucleic acid further comprises the chemically-modified nucleotide at one or more of positions 1, 3, 5, 20, 22 and 24 of the nucleotide sequence. In some embodiments, the chemically-modified nucleotide is a locked nucleic acid (LNA). In some embodiments, the nucleotide sequence comprises the LNA at positions 1, 3, 5, 20, 22 and 24 of the nucleotide sequence. In some embodiments, the nucleic acid is at most 30 nt long.

In some embodiments, nucleic acid consists of or consists essentially of the nucleotide sequence: CGUCCGAUGGUAGUGGGUUAUCAG.

A nucleic acid consisting of a nucleotide sequence: CGUCCGAUGGUAGUGGGUUAUCAG (SEQ ID NO: 2), wherein the nucleic acid is RNA, wherein each of positions 1, 3, 5, 20, 22 and 24 of the nucleotide sequence is a LNA.

Also provided is a composition comprising: any one of the isolated nucleic acids of the present disclosure; and a pharmaceutically acceptable excipient. Optionally, the composition further comprises a transfection reagent. Optionally, the transfection reagent comprises one or more of a liposome, an extracellular vesicle (EV), and a polyethylene glycol (PEG)-cationic lipid complex (PCLC). In some embodiments, the transfection reagent comprises EV derived from cardiosphere-derived cells (CDC). In some embodiments, the composition further comprises a casein phosphoprotein. Optionally, the composition further comprises chitosan. Optionally, the isolated nucleic acid is encapsulated in a casein-chitosan complex. In some embodiments, the composition comprises casein micelles.

Provided herein is a macrophage exposed to, or transfected with, any one of the nucleic acids of the present disclosure, wherein an anti-inflammatory activity of the macrophage is increased compared to a macrophage without the nucleic acid. Optionally, the macrophage is in a subject. Optionally, the macrophage is in culture.

Also provided is a kit comprising: any one of the nucleic acids of the present disclosure; and a transfection reagent. Optionally, the transfection reagent is one or more of a liposome, an extracellular vesicle (EV), and a polyethylene glycol (PEG)-cationic lipid complex (PCLC). In some embodiments, the kit further comprises a pharmaceutically acceptable excipient. In some embodiments, the kit further comprises a casein phosphoprotein. In some embodiments, the kit further comprises chitosan.

Also provided is a method of treating a heart condition or symptom thereof, comprising administering to a subject in need of treating a heart condition or symptom thereof a therapeutically effective amount of any one of the nucleic acids of the present disclosure or any one of the composition of the present disclosure, thereby treating the heart condition or symptom thereof. Optionally, the heart condition comprises a symptom and/or sequelae of heart failure. In some embodiments, the heart condition comprises hypertrophic cardiomyopathy. In some embodiments, the heart condition comprises heart failure with preserved ejection fraction (HFpEF). In some embodiments, the heart condition comprises a symptom or sequelae of an infectious disease. In some embodiments, the infectious disease comprises a viral infection. In some embodiments, the subject has the heart condition. In some embodiments, the subject is at risk of developing the heart condition. In some embodiments, the subject exhibits, before the administering, one or more of: hypertension, elevated E/e′ ratio by echocardiography, cardiac hypertrophy, myocardial fibrosis, obesity, wasting, reduced endurance, and elevated systemic inflammatory markers.

Also provided is a method treating a muscle disorder or symptom thereof, comprising administering to a subject in need of treating a muscle disorder or symptom thereof a therapeutically effective amount of any one of the nucleic acids of the present disclosure or any one of the compositions of the present disclosure, thereby treating the muscle disorder or symptom thereof. Optionally, the muscle disorder comprises muscular dystrophy or a heart condition. In some embodiments, the muscle disorder comprises Duchenne muscular dystrophy. In some embodiments, the subject has the muscle disorder. In some embodiments, the subject is at risk of developing the muscle disorder. In some embodiments, the subject is genetically predisposed to developing the muscle disorder. In some embodiments, the subject exhibits, before the administering, one or more of: reduced endurance, and reduced skeletal muscle function.

Also provided is a method of treating an inflammatory condition, comprising administering to a subject in need of treating an inflammatory condition a therapeutically effective amount of any one of the nucleic acids of the present disclosure or any one of the compositions of the present disclosure, thereby treating the inflammatory condition. Optionally, the inflammatory condition comprises a symptom or sequelae of an infectious disease. In some embodiments, the infectious disease comprises a viral infection. In some embodiments, the inflammatory condition comprises a cytokine storm. In some embodiments, the inflammatory condition is associated with immunotherapy (e.g., for cancer). In some embodiments, the inflammatory condition is scleroderma, an autoimmune condition affecting the skin. In some embodiments, the inflammatory condition is systemic sclerosis, an autoimmune condition resembling scleroderma but affecting not only the skin but also internal organs including the lung and the heart.

Also provided is a method of treating a fibrotic condition, comprising administering to a subject in need of treating a fibrotic condition a therapeutically effective amount of any one of the nucleic acids of the present disclosure or any one of the compositions of the present disclosure, thereby treating the fibrotic condition. Optionally, the fibrotic condition comprises a symptom or sequelae of an infectious disease. In some embodiments, the infectious disease comprises a viral infection. In some embodiments, the fibrotic condition is idiopathic pulmonary fibrosis. In some embodiments, the fibrotic condition is cirrhosis of the liver.

In some embodiments, the therapeutically effective amount of the nucleic acid comprises from about 0.001 μg/g to about 100 μg/g. In some embodiments, any one of the treatment methods of the present disclosure includes administering the therapeutically effective amount of the nucleic acid or the composition no more frequently than twice a week. In some embodiments, the method includes administering the therapeutically effective amount of the nucleic acid or the composition intravenously, intramuscularly, intracardially, or orally. Optionally, the therapeutically effective amount of the nucleic acid or the composition is administered orally.

Also provided is a method of promoting anti-inflammatory activity of macrophages, comprising contacting any one of the nucleic acids of the present disclosure or any one of the compositions of the present disclosure with a population of macrophages, to thereby promote an anti-inflammatory activity of macrophages of the population. Optionally, the contacting comprises administering to a subject in need of treating a condition characterized by inflammation or fibrosis an effective amount of the nucleic acid or the composition, to thereby promote an anti-inflammatory activity of macrophages in the subject. In some embodiments, the macrophage is a human macrophage.

### BRIEF DESCRIPTION OF THE DRAWINGS

FIGS. 1A-1B show various non-limiting schematics for oral formulations as provided for herein. FIG. 1A shows a non-limiting schematic in which a lipid is used to encapsulate a nucleic acid, such as a therapeutic nucleic acid, and the encapsulated nucleic acid is coated with a casein-chitosan complex, which allows for oral delivery, FIG. 1B shows alternative non-limiting schematics of alternative formulations for therapeutic nucleic acids, such as RNA (e.g., non-coding RNA or coding RNA).

FIGS. 2A-2C show schematics and data related to oral formulations as provided for herein. FIGS. 2A 2B show various non-limiting schematics of non-coding RNAs (TY4 in 2A and piR-659/piREX1 in 2B) and their encapsulation in lipid-casein-chitosan complexes according to embodiments disclosed herein. FIG. 2C shows data demonstrating that oral administration of piREX1 (and a derivative thereof, U to A) are effective in reducing infarct mass and cardiac troponin levels (as compared to control).

FIGS. 3A-3M show the therapeutic effects of administering TY4 in heart failure with preserved ejection fraction (HFpEF), according to some non-limiting embodiments of the present disclosure. FIG. 3A is a schematic diagram showing a protocol for measuring the therapeutic effect of TY4 in a model of HFpEF. FIG. 3B is a graph comparing systolic blood pressure (left panel) and diastolic blood pressure (right panel). FIG. 3C is a graph comparing treadmill exercise distances. FIG. 3D is a graph comparing the ratio of early diastolic mitral inflow velocity to mitral annular tissue velocity (E/e′). FIG. 3E is a graph comparing systemic brain natriuretic peptide (BNP) levels. FIG. 3F is a collection of graphs comparing systolic blood pressure (left panel) and diastolic blood pressure (right panel). FIG. 3G is a graphs comparing the ratio of early diastolic mitral inflow velocity to mitral annular tissue velocity (E/e′). FIG. 3H is a graph comparing treadmill exercise distances. FIG. 3I is a graph comparing systemic BNP levels. FIG. 3J is a graph comparing systolic blood pressure over time. FIG. 3K is a graph comparing diastolic blood pressure over time. FIG. 3L is a graph comparing E/c′ ratios over time. FIG. 3M is a graph comparing treadmill exercise distances over time.

FIGS. 4A-4F show the therapeutic effects of administering TY4 in heart failure with preserved ejection fraction (HFpEF), according to some non-limiting embodiments of the present disclosure. The data of FIGS. 4A-4E are from additional replicates of the corresponding data from FIG. 3 (e.g., greater sample size). FIG. 4A is a graph comparing systolic blood pressure in animals receiving control, vehicle, and oral formulations. FIG. 4B is a graph comparing diastolic blood pressure in animals receiving control, vehicle, and oral formulations. FIG. 4C is a graph comparing the ratio of early diastolic mitral inflow velocity to mitral annular tissue velocity (E/e′) in animals receiving control, vehicle, and oral formulations. FIG. 4D is a graph comparing is a graph comparing systemic brain natriuretic peptide (BNP) levels in animals receiving control, vehicle, and oral formulations. FIG. 4E is a graph comparing treadmill exercise distances in animals receiving control, vehicle, and oral formulations. FIG. 4F is a graph comparing the circulating blood glucose levels in animals receiving control, vehicle, and oral formulations. FIG. 4G shows cardiac size for animals receiving control, vehicle, and oral formulations.

FIGS. 5A-5E show data related to efficacy of oral administration of therapeutic RNA in a model of myocardial infarction. FIG. 5A shows data related to infarct size. FIG. 5B shows cardiac tissue sections from the various treatment groups. FIG. 5C shows data for circulating levels of cardiac troponin 48 hours post-injury. FIG. 5D shows additional data related to infarct mass, including a treatment group in which chitosan was not used in the oral formulation. FIG. 5E shows additional data related circulating cardiac troponin levels, including a treatment group in which chitosan was not used in the oral formulation.

FIGS. 6A-6E show data related to oral formulations delivering therapeutic nucleic acids in a model of scleroderma. FIG. 6A shows data related to distance traveled on a treadmill in the indicated treatment groups. FIG. 6B shows data related to body weight. FIG. 6C shows data related to the ratio of heart weight to body weight, representing a heart index (HI). FIG. 6d shows data related to the ratio of lung weight to body weight, representing a pulmonary index (PI). FIG. 6E shows data related to lung weight for the indicated groups.

FIGS. 7A-7D show additional data related to oral formulations delivering therapeutic nucleic acids in a model of scleroderma. FIG. 7A shows histology data related to cardiac fibrosis. FIG. 7B shows a summary of cardiac fibrosis data, tabulated by treatment group. FIG. 7C shows histology related to skin fibrosis. FIG. 7D shows a summary of skin fibrosis data, tabulated by treatment group.

FIGS. 8A-8F shows additional data related to scleroderma. The data depicted show quantification of IL1-B (8A), IL-6 (8B), TGF beta (8C), NLRP3 (8D), p21 (8E), and IL-4 (8F).

FIGS. 9A-9H show data related to oral formulations delivering therapeutic nucleic acids in a model of muscular dystrophy. FIG. 9A shows data related to transthoracic echocardiography to measure left ventricular ejection fraction (EF). FIG. 9B shows Masson's trichrome micrographs and pooled data (right subpanel) showing that oral administration of a therapeutic nucleic acid results in less myocardial fibrosis than vehicle control mice. FIG. 9C shows data summarizing muscle function at baseline and after 8 weeks. FIG. 9D shows Masson's trichrome micrographs and pooled data (right subpanel) showing that oral administration of a therapeutic nucleic acid results in less muscle fibrosis than vehicle control mice. FIG. 9E shows data related to the myofiber count in control versus orally delivered therapeutic RNA. FIG. 9F shows data related to the exercise capacity of orally treated animals versus control. FIG. 9G shows data related to the cardiac function of orally treated animals versus control. FIG. 9H shows data related to the muscle function of orally treated animals versus control.

### DETAILED DESCRIPTION

Nucleic acid therapeutics offer the potential to treat diseases at a genetic level. Many conventional treatments generally induce therapeutic effects that are transient because they target proteins rather than underlying causes. In contrast, nucleic acid therapeutics have the potential for long-lasting (or even permanent, e.g., curative) effects via gene inhibition, addition, replacement, or editing. However, the successful use of nucleic acid therapeutics will hinge on delivery technologies that improve stability and/or bioavailability.

In several embodiments, the formulations provided for herein allow the enhanced delivery of nucleic acids to a subject. In several embodiments, the nucleic acids comprise DNA. In several embodiments, the nucleic acids comprise RNA. In several embodiments, the nucleic acids comprise coding RNA (e.g., messenger RNA or mRNA). In several embodiments, the nucleic acids comprise non-coding RNAs (ncRNA). mRNA can provide therapeutic effects by virtue of delivery of a sequence coding for a protein that yields a therapeutic effect. Depending on the embodiment, that could be a protein that replaces a non-functional protein. In other embodiments, the coding RNA could encode a protein to which an immune response is desired (e.g., a viral protein), such as for the production of antibodies against that protein. ncRNA are known to exhibit positive therapeutic effects based on their ability to increase secretion of anti-inflammatory cytokines or decrease secretion of inflammatory cytokines. Certain ncRNA exhibit cardioprotective, anti-fibrotic, and/or anti-hypertrophic effects.

Prior to the disclosure provided herein, ncRNA were delivered to a subject in need thereof via a parenteral administration route, such as injection intramuscularly, subcutaneously, or via intravenous administration. Provided herein, in several embodiments, are formulations that allow for the oral delivery of ncRNA, or other therapeutic nucleic acids. In several embodiments, the formulations protect the nucleic acid from the low acid environment of the upper/mid GI tract (e.g., the stomach) such that they reach the lower GI tract in an intact form and are absorbed in a form that allows their anti-inflammatory and/or anti-fibrotic effects to be realized.

In several embodiments, the formulations provided for herein allow the use of nucleic acids in treating conditions where inflammation and/or tissue injury are the main drivers of pathology. In some embodiments, conditions treated using such formulations, include, without limitation, inflammatory disease, muscular dystrophy, or cardiac injury. In some embodiments, the formulations have cardioprotective effects when administered to a subject suffering from cardiac injury due to, without limitation, myocardial infarction and/or heart failure, among other maladies, such as scleroderma and/or muscular dystrophy. Without being bound by theory, the compositions of the present disclosure allow the successful delivery of nucleic acids, such as ncRNA, to the lower GI tract, where they are readily absorbed and can increase an anti-inflammatory activity of macrophages, e.g., by promoting secretion of interleukin 10 (IL-10) from macrophages. In some embodiments, the compositions of the present disclosure allow the successful delivery of nucleic acids, such as ncRNA to the lower GI tract, where they are readily absorbed and can induce changes in expression of one or more gene products and/or epigenetic changes in macrophages that are exposed to the nucleic acids. In some embodiments, conditions, e.g., inflammatory disease, muscular dystrophy, or cardiac injury, treated by administration of formulations comprising nucleic acids of the present disclosure.

### Definitions

As used herein the term “nucleic acid” or “oligonucleotide” refers to multiple nucleotides (e.g., molecules comprising a sugar (e.g. ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). The term includes polynucleosides (i.e. a polynucleotide minus the phosphate) and any other organic base containing polymer. Purines and pyrimidines include but are not limited to adenine, cytosine, guanine, thymidine, inosine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. A nucleic acid can include any other suitable modifications. Thus, the term nucleic acid also encompasses nucleic acids with substitutions or modifications, such as in the bases and/or sugars.

Polypeptide or nucleic acid molecules of the present disclosure may share a certain degree of sequence similarity or identity with the reference molecules (e.g., reference polypeptides or reference polynucleotides), for example, with art-described molecules (e.g., engineered or designed molecules or wild-type molecules). The term “identity” as known in the art, refers to a relationship between the sequences of two or more polypeptides or polynucleotides, as determined by comparing the sequences. In the art, identity also means the degree of sequence relatedness between them as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Any suitable methods and computer programs for the alignment can be used. It is understood that identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”. Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197.) A general global alignment technique based on dynamic programming is the Needleman-Wunsch algorithm (Needleman, S. B. & Wunsch. C. D. (1970) “A general method applicable to the search for similarities in the amino acid sequences of two proteins.” J. Mol. Biol. 48:443-453.). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm. Other tools are described herein, specifically in the definition of “identity” below.

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a suitable mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects. Smith. D. W., ed., Academic Press. New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM 120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

The term “Watson-Crick base-pairing”, or “base-pairing” refers to the formation of hydrogen bonds between specific pairs of nucleotide bases (“complementary base pairs”). For example, two hydrogen bonds form between adenine (A) and uracil (U), and three hydrogen bonds form between guanine (G) and cytosine (C). One method of assessing the strength of bonding between two polynucleotides is by quantifying the percentage of bonds formed between the guanine and cytosine bases of the two polynucleotides (“GC content”). In some embodiments, the GC content of bonding between two nucleic acids of a multimeric molecule (e.g., a multimeric mRNA molecule) is at least 10%, at least 20%, at least 30%, at least 40%, or at least 50%. In some embodiments, the GC content of bonding between two nucleic acids of a multimeric molecule (e.g., a multimeric mRNA molecule) is between 10% and 70%, about 20% to about 60%, or about 30% to about 60%. The formation of a nucleic acid duplex via bonding of complementary base pairs can also be referred to as “hybridization”. Generally, two nucleic acids sharing a region of complementarity are capable, under suitable conditions, of hybridizing (e.g., via nucleic acid base pairing) to form a duplex structure. A region of complementarity can vary in size. In some embodiments, a region of complementarity ranges in length from about 2 base pairs to about 100 base pairs. In some embodiments, a region of complementarity ranges in length from about 5 base pairs to about 75 base pairs. In some embodiments, a region of complementarity ranges in length from about 10 base pairs to about 50 base pairs. In some embodiments, a region of complementarity ranges in length from about 20 base pairs to about 30 base pairs.

“Isolated” as used herein with reference to an isolated biomolecule, e.g., a nucleic acid, has the ordinary and customary meaning to one of ordinary skill in the art in view of the present disclosure. An isolated biomolecule, e.g., an isolated nucleic acid, is generally in a non-natural environment, or in an environment that the biomolecule would otherwise not have been without human intervention of the biomolecule or its environment. In some embodiments, an isolated biomolecule is not inside a cell or an organism.

“Extracellular vesicle” or “EV” as used herein have their ordinary and customary meaning as understood by one of ordinary skill in the art, in view of the present disclosure. EVs include lipid bilayer structures generated by cells, and include exosomes, microvesicles, epididimosomes, argosomes, exosome-like vesicles, microparticles, promininosomes, prostasomes, dexosomes, texosomes, dex, tex, archeosomes and oncosomes.

“Micelle.” as used herein with reference to casein micelles, has its customary and ordinary meaning as understood by one of ordinary skill in the art, in view of the present disclosure. Casein micelles are colloidal particles that can include aggregates of one or more casein phosphoproteins (e.g., one or more, two or more, three or more, or all four of alpha s1 casein, alpha s2 casein, beta casein, and kappa casein).

“Subject.” as used herein refers to any vertebrate animal, including mammals and non-mammals. A subject can include primates, including humans, and non-primate mammals, such as rodents, domestic animals or game animals. Non-primate mammals can include mouse, rat, hamster, rabbit, dog, fox, wolf, cat, horse, cow, pig, sheep, goat, camel, deer, buffalo, bison, etc. Non-mammals can include bird (e.g., chicken, ostrich, emu, pigeon), reptile (e.g., snake, lizard, turtle), amphibian (e.g., frog, salamander), fish (e.g., salmon, cod, pufferfish, tuna), etc. The terms, “individual,” “patient.” and “subject” are used interchangeably herein.

“Administering” as used herein can include any suitable routes of administering a therapeutic agent or composition as disclosed herein. Suitable routes of administration include, without limitation, oral, parenteral, intravenous, intramuscular, subcutaneous, transdermal, airway (aerosol), pulmonary, cutaneous, injection or topical administration. Administration can be local or systemic.

As used herein, “treat” and “treatment” includes curing, improving, ameliorating, reducing the severity of, preventing, slowing the progression of, and/or delaying the appearance of a disease, condition and/or symptoms thereof.

A treatment can be considered “effective,” or “therapeutically effective” as used herein, if one or more of the signs or symptoms of a condition described herein are altered in a beneficial manner, other clinically accepted symptoms are improved, or even ameliorated, or a desired response is induced e.g., by at least 2%, 3%, 4%, 5%, 10%, or more, following treatment according to the methods described herein. Efficacy can be assessed, for example, by measuring a marker, indicator, symptom, and/or the incidence of a condition treated according to the methods described herein or any other measurable parameter appropriate, e.g. exercise endurance. Efficacy can also be measured by a failure of an individual to worsen as assessed by hospitalization, or need for medical interventions (e.g., progression of the disease is halted). Treatment includes any treatment of a disease or condition in an individual or an animal (some non-limiting examples include a human or an animal) and includes: (1) inhibiting the disease or condition, e.g., preventing a worsening of symptoms (e.g. pain or inflammation); or (2) relieving the severity of the disease or condition, e.g., causing regression of symptoms. An effective amount for the treatment of a disease or condition means that amount which, when administered to a subject in need thereof, is sufficient to result in effective treatment as that term is defined herein, for that disease or condition. Efficacy of an agent can be determined by assessing physical indicators of a condition or desired response, (e.g. muscle function, mass or volume). One skilled in the art can monitor efficacy of administration and/or treatment by measuring any one of such parameters, or any combination of parameters.

The term “effective amount” or “therapeutically effective amount” as used herein refers to the amount of a composition or an agent needed to alleviate at least one or more symptom of the disease or condition, and relates to a sufficient amount of therapeutic composition to provide the desired effect. The term “effective amount” or “therapeutically effective amount” can refer to an amount of a composition or therapeutic agent that is sufficient to provide a particular anti-inflammatory and/or cardioprotective effect when administered to a typical subject. An effective amount as used herein, in various contexts, can include an amount sufficient to delay the development of a symptom of the disease or condition, alter the course of a symptom disease or condition (for example but not limited to, slowing the progression of a symptom of the disease or condition), or reverse a symptom of the disease or condition. In some embodiments, the therapeutically effective amount is administered in one or more doses of the therapeutic agent. In some embodiments, the therapeutically effective amount is administered in a single administration, or over a period of time in a plurality of doses.

As used herein, the phrase “physiologically compatible” and “pharmaceutically acceptable” are employed interchangeably herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Definitions of common terms in cell biology and molecular biology can be found in “The Merck Manual of Diagnosis and Therapy”. 19th Edition, published by Merck Research Laboratories, 2006 (ISBN 0-91 1910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009. Wiley Intersciences, Coligan et al., eds.

The singular terms “a,” “an,” and “the” include plural referents unless context clearly indicates otherwise. Similarly, the word “or” is intended to include “and” unless the context clearly indicates otherwise. The abbreviation, “e.g.” is used herein to indicate a non-limiting example. Thus, the abbreviation “e.g.” is synonymous with the term “for example.” The term “about” as used herein to, for example, define the values and ranges of molecular weights means that the indicated values and/or range limits can vary within +20%, e.g., within +10%, including within +5%. The use of “about” before a number includes the number itself. For example, “about 5” provides express support for “5.” Numbers provided in ranges include overlapping ranges and integers in between; for example a range of 1-4 and 5-7 includes for example, 1-7, 1-6, 1-5, 2-5, 2-7, 4-7, 1, 2, 3, 4, 5, 6 and 7.

### Compositions

In several embodiments, provided herein are compositions that are configured for oral administration and comprise a therapeutic nucleic acid (e.g., an RNA (coding or non-coding RNA) molecule encapsulated by the composition). In several embodiments, the composition is a pharmaceutical composition. In some embodiments the composition includes pharmaceutically acceptable excipient. In some embodiments, the composition is a cell-free composition, e.g., the composition is substantially free of cells such as CDC. In several embodiments, the compositions is free of cell-derived materials, such as exosomes or cellular vesicles. In several embodiments, the composition comprises an artificial vesicle (e.g., a vesicle formed from lipids, such as cationic lipids).

Some non-limiting examples of materials which can serve as pharmaceutically-acceptable excipients include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) is otonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations.

In some embodiments, the composition includes a transfection reagent, e.g., to promote delivery of the nucleic acid to a target cellular target (in vitro or in vivo). Any suitable transfection reagent can be included in the composition. Suitable transfection reagents include, without limitation, a liposome, extracellular vesicle (EV), and a polyethylene glycol (PEG)-cationic lipid complex (PCLC). In several embodiments, a cationic lipid is used. In some embodiments, the transfection reagent includes DharmaFECT® or Lipofectamine®. In several embodiments, the transfection reagent comprises DharmaFECT®. In some embodiments, the nucleic acid of the present disclosure is formulated with the transfection reagent in the composition so as to promote cellular uptake and/or pharmacokinetics of the nucleic acid.

Liposomes are artificially-prepared vesicles which may primarily be composed of a lipid bilayer (or spherical monolayer oriented such that the hydrophobic tails of the lipids are positioned within the sphere) and may be used as a delivery vehicle for the administration of pharmaceutical formulations. Liposomes can be of different sizes such as, but not limited to, a multilamellar vesicle (MLV), which may be hundreds of nanometers in diameter and may contain a series of concentric bilayers separated by narrow aqueous compartments, a small unicellular vesicle (SUV), which may be smaller than 50 nm in diameter, and a large unilamellar vesicle (LUV), which may be between 50 and 500 nm in diameter. Liposome design may include, without limitation, opsonins or ligands in order to improve the attachment of liposomes to target tissue/cells, or to activate events such as, but not limited to, endocytosis. Liposomes may contain a low or a high pH in order to improve the delivery of the cargo, e.g., a nucleic acid of the present disclosure.

In some embodiments, the composition includes, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminocthyl)-[1,3]-dioxolane (DLin-KC2-DMA), and MC3 and liposomes such as, but not limited to, DOXIL® from Janssen Biotech, Inc. (Horsham, Pa.).

In some embodiments, the composition includes a cationic lipid. Any suitable cationic lipid may be used in the present compositions. Suitable cationic lipids include, without limitation, DLin-DMA, DLin-D-DMA, DLin-MC3-DMA, DLin-KC2-DMA, DODMA and amino alcohol lipids. In some embodiments, the composition includes a cationic lipid complex, e.g., a polyethylene glycol (PEG)-cationic lipid complex (PCLC). In some embodiments, the cationic lipid is PEGylated, e.g., 2 kDa PEG (“PEG2000”). Any suitable option can be used to PEGylate the cationic lipid. In some embodiments, PCLC is formed by exposing a mixture of PEG and the cationic lipid to one or more freeze/thaw cycles, e.g., 1, 2, 3, 4, 5 or more freeze/thaw cycles. In some embodiments, a freeze/thaw cycle includes freezing the mixture with liquid nitrogen (e.g., around −190° C.) for about 5 minutes, and thawing at about 60° C. for about 5 minutes. A nucleic acid of the present disclosure can be mixed with the PCLC to generate a complex of the nucleic acid and the PCLC.

In some embodiments, the composition includes casein, e.g., a casein micelle. In some embodiments, the composition includes chitosan. In some embodiments, the composition includes casein and chitosan. In some embodiments, the composition includes a casein-chitosan complex. In some embodiments, the isolated nucleic acid in the composition is encapsulated in a casein-chitosan complex. In some embodiments, the composition includes one or more of phosphoproteins: alpha s1 casein, alpha s2 casein, beta casein, and kappa casein. In some embodiments, the composition includes two or more, three or more, or all four phosphoproteins: alpha s1 casein, alpha s2 casein, beta casein, and kappa casein. The phosphoproteins may be present in the composition at any suitable concentration (relative to each other, and relative to the total volume of the composition), and in some embodiments, is present in an amount suitable for forming casein micelles. In some embodiments, the casein phosphoproteins are collectively present in the composition at about 5-10% (weight by volume). Unless otherwise indicated, the use of the term weight per volume or weight by volume assumes that 1 g/100 mL=1% w/v. In some embodiments, the casein phosphoproteins are collectively present in the composition at about 8% (weight by volume). The casein phosphoproteins can be those from any suitable animal, e.g., mammal such as, but not limited to, human, non-human primate, cow, pig, horse, camel, goat, and sheep. In some embodiments, the casein phosphoproteins are bovine alpha s1 casein, alpha s2 casein, beta casein, and kappa casein. Suitable casein formulations with EV are provided in, e.g., Aminzadeh et al., J Extracell Vesicles. 2021 January; 10(3):e12045, the entirety of which is incorporated herein by reference. In some embodiments, a composition, e.g., pharmaceutical composition, of the present disclosure formulated with casein, as provided herein, is suitable for oral administration to the subject.

In several embodiments, a composition for enhancing the oral bioavailability of a therapeutic nucleic acid of the present disclosure comprises at least two phosphoproteins selected from alpha s1 casein, alpha s2 casein, beta casein, and kappa casein, where the phosphoproteins are present in an amount between about 5% to about 10% (weight by volume) of the composition, in a physiologically compatible excipient. In several embodiments, the composition includes the alpha s1 casein in an amount between about 0% to about 50% (e.g., about 10% to about 45%, about 20% to about 40%, about 25% to about 40%, about including 30% to about 40%) (by weight), the alpha s2 casein in an amount between about 0% to about 20% (e.g., about 5% to about 15%, about 7% to about 12%, including about 8% to about 12%) (by weight), the beta casein in an amount between about 0% to about 50% (e.g., about 10% to about 45%, about 20% to about 40%, about 25% to about 40%, about including 30% to about 40%) (by weight), and the kappa casein in an amount between about 0% to about 20% (e.g., about 5% to about 18%, about 8% to about 18%, including about 10% to about 15%) (by weight) of the phosphoprotein mass in the composition. The present compositions can provide for enhanced oral bioavailability of therapeutic nucleic acids, such as non-coding RNA. In several embodiments, the therapeutic nucleic acid comprises RNA, such as, but not limited to mRNA and non-coding RNA (e.g., miRNA, lncRNA). In some embodiments, the payload is a synthetic molecule, e.g., a small molecule or drug.

In several embodiments, the formulations provided for herein are in the form of lipid-bound vesicles, e.g., micelles or liposomes, and can therefore include any suitable number of particles. In some embodiments, the amount of micelles (e.g., casein-chitosan coated micelles) is in a range of about 10⁶ to about 10¹⁰ particles, e.g., about 2×10⁶ to about 10¹⁰ particles, about 5×10⁶ to about 10¹⁰ particles, about 10⁷ to about 5×10⁹ particles, about 2×10⁷ to about 5×10⁹ particles, about 5×10⁷ to about 5×10⁹ particles, including about 1×10⁸ to about 2×10⁹ particles. In some embodiments, the amount of micelles (e.g., casein-chitosan coated micelles) in the population is about 10⁶, about 2×10⁶, about 5×10⁶, about 10⁷, about 2×10⁷, about 5×10⁷, about 10⁸, about 2×10⁸, about 5×10⁸, about 10⁹, about 2×10⁹, about 5×10⁹, or about 10¹⁰ particles, or an amount in between any two of the preceding values.

In several embodiments, the composition comprises casein-chitosan coated lipid micelles, where the casein phosphoproteins are present in the composition in suitable amounts (e.g., suitable total amount of phosphoprotein mass in the composition, suitable proportions of phosphoproteins relative to each other). In some embodiments, the composition includes two, three, or all four phosphoproteins selected from alpha s1 casein, alpha s2 casein, beta casein, and kappa casein. In some embodiments, the amount of a phosphoprotein in the composition depends on the amount of one or more other phosphoprotein present in the composition.

In some embodiments, alpha s1 casein is a phosphoprotein associated with the gene name CSN1S1. The alpha s1 casein can be a CSN1S1 phosphoprotein from any suitable mammal. In some embodiments, the alpha s1 casein is bovine (Gene ID: 282208), porcine (Gene ID: 445514), equine (Gene ID: 100033982), ovine (Gene ID: 443382), caprine (Gene ID: 100750242), cameline (Gene ID: 105090954), or human (Gene ID: 1446). In some embodiments, the alpha s1 casein is a non-human alpha s1 casein. In some embodiments, the alpha s1 casein is a polypeptide having an amino acid sequence at least 80%, at least 85%, at least 90%, at least 95%, at least 98%, or about 100% identical to the sequence set forth in SEQ ID NO: 26.

In some embodiments, the composition includes any suitable amount of alpha s1 casein. In some embodiments, the composition includes the alpha s1 casein in an amount, by weight, between about 0% to about 50%, e.g., between about 5% to about 50%, between about 10% to about 50%, between about 15% to about 45%, between about 20% to about 45%, including between about 25% to about 40%, of the phosphoprotein mass in the composition. In some embodiments, the composition includes the alpha s1 casein in an amount, by weight, of about 0%, 5%, 10%, 15%, 20%, 25%, 26%, 27%, 28%, 29%, 30%, 31%, 32%, 33%, 34%, 35%, 36%, 37%, 38%, 39%, 40%, or an amount within a range defined by any two of the preceding values.

In some embodiments, the alpha s2 casein is a phosphoprotein associated with the gene name CSN1S2. The alpha s2 casein can be a CSN1S2 phosphoprotein from any suitable mammal. In some embodiments, the alpha s2 casein is bovine (Gene ID: 282209), porcine (Gene ID: 445515), equine (Gene ID: 100327035), ovine (Gene ID: 443383), caprine (Gene ID: 100861229), or cameline (Gene ID: 105090951). In some embodiments, the alpha s2 casein is a polypeptide having an amino acid sequence at least 80%, at least 85%, at least 90%, at least 95%, at least 98%, or about 100% identical to the sequence set forth in SEQ ID NO: 27.

The composition can include any suitable amount of alpha s2 casein. In some embodiments, the composition includes the alpha s2 casein in an amount, by weight, between about 0% to about 20%, e.g., between about 2% to about 18%, between about 3% to about 18%, between about 4% to about 17%, between about 5% to about 16%, including between about 5% to about 15%, of the phosphoprotein mass in the composition. In some embodiments, the composition includes the alpha s2 casein in an amount, by weight, of about 0%, 1%, 2%, 3%, 4%, 5%, 6%, 7%, 8%, 9%, 10%, 11%, 12%, 13%, 14%, 15%, 18%, 20%, or an amount within a range defined by any two of the preceding values.

In some embodiments, the beta casein is a phosphoprotein associated with the gene name CSN2. The beta casein can be a CSN2 phosphoprotein from any suitable mammal. In some embodiments, the beta casein is bovine (Gene ID: 281099), porcine (Gene ID: 404088), equine (Gene ID: 100033903), ovine (Gene ID: 443391), caprine (Gene ID: 100860784), cameline (Gene ID: 105080412), or human (Gene ID: 1447). In some embodiments, the beta casein is a non-human beta casein. In some embodiments, the beta casein is a polypeptide having an amino acid sequence at least 80%, at least 85%, at least 90%, at least 95%, at least 98%, or about 100% identical to the sequence set forth in SEQ ID NO: 28 or 29.

The composition can include any suitable amount of beta casein. In some embodiments, the composition includes the beta casein in an amount, by weight, between about 0% to about 50%, e.g., between about 5% to about 50%, between about 10% to about 50%, between about 15% to about 45%, between about 20% to about 45%, including between about 25% to about 40%, of the phosphoprotein mass in the composition. In some embodiments, the composition includes the beta casein in an amount, by weight, of about 0%, 5%, 10%, 15%, 20%, 25%, 26%, 27%, 28%, 29%, 30%, 31%, 32%, 33%, 34%, 35%, 36%, 37%, 38%, 39%, 40%, or an amount within a range defined by any two of the preceding values.

In some embodiments, the kappa casein is a phosphoprotein associated with the gene name CSN3. The beta casein can be a CSN3 phosphoprotein from any suitable mammal. In some embodiments, the kappa casein is bovine (Gene ID: 281728), porcine (Gene ID: 445511), equine (Gene ID: 100033983), ovine (Gene ID: 443394), caprine (Gene ID: 100861231), cameline (Gene IDs: 105080408 or 105090949), or human (Gene ID: 1448). In some embodiments, the kappa casein is a non-human kappa casein. In some embodiments, the kappa casein is a polypeptide having an amino acid sequence at least 80%, at least 85%, at least 90%, at least 95%, at least 98%, or about 100% identical to the sequence set forth in SEQ ID NO: 30.

The composition can include any suitable amount of kappa casein. In some embodiments, the composition includes the kappa casein in an amount, by weight, between about 0% to about 20%, e.g., between about 2% to about 18%, between about 3% to about 18%, between about 4% to about 17%, between about 5% to about 16%, including between about 5% to about 15%, of the phosphoprotein mass in the composition. In some embodiments, the composition includes the kappa casein in an amount, by weight, of about 0%, 1%, 2%, 3%, 4%, 5%, 6%, 7%, 8%, 9%, 10%, 11%, 12%, 13%, 14%, 15%, 18%, 20%, or an amount within a range defined by any two of the preceding values.

Combinations of caseins from different species are used, in some embodiments. For example, in several embodiments, one or more human casein is used in combination with one or more bovine casein. Ratios of caseins are used in some embodiments, for example a 3:1:3:1 ratio of alpha S1 casein:alpha s2 casein:beta casein:kappa casein. Different ratios may be used in some embodiments, for example 4:1:4:1, 2:1:2:1, or 1:1:1:1. Ratios may also be used between any two given caseins in a composition, ranging from 1:1, 2:1, 3:1, 4:1, 5:1, 10:1, 1:5, 1:4, 1:3, 1:2, etc.

Any suitable total amount of the phosphoproteins may be present in the composition. In some embodiments, the phosphoproteins are present in an amount between 5% to about 10%, e.g., about 6% to about 10%, about 6% to about 9%, including about 6% to about 8%, (weight by volume) of the composition. In some embodiments, the phosphoproteins are present in an amount of about 5%, 6%, 7%, 8%, 9%, 10%, or an amount within a range defined by any two of the preceding values, (weight by volume) of the composition.

In some embodiments, one or more of the casein phosphoproteins are non-human casein phosphoproteins. In some embodiments, the exosomes and at least one of the casein phosphoproteins are from different species. In some embodiments, the exosomes are human exosomes, and one or more of the casein phosphoproteins are non-human casein phosphoproteins. In some embodiments, the exosomes are human exosomes, and one or more of the casein phosphoproteins are bovine (or ovine, porcine, caprine, cameline, or equine) casein phosphoproteins.

In some embodiments, the composition includes micellar structures formed by at least a portion of the casein phosphoproteins. In some embodiments, the casein micelles are substantially spherical. In some embodiments, a casein micelle in the composition has an average diameter (as measured per micelle) of about 40 nm, about 50 nm, about 60 nm, about 70 nm, about 80 nm, about 90 nm, about 100 nm, about 110 nm, about 120 nm, about 130 nm, about 150 nm, about 200 nm, about 250 nm, about 300 nm, about 350 nm, about 400 nm, about 450 nm, about 500 nm or more, or an average diameter within a range defined by any two of the preceding values. In some embodiments, a casein micelle in the composition has an average diameter (as measured per micelle) in a range from about 40 nm to about 500 nm, e.g., from about 40 nm to about 400 nm, from about 50 nm to about 300 nm, from about 60 nm to about 250 nm, from about 70 nm to about 250 nm, from about 80 nm to about 200 nm, including from about 90 nm to about 150 nm. The casein micelles of the present composition are generally not precipitated or in gel form.

In some embodiments, the composition includes one or more colloidal minerals (e.g., minerals in suspension). In several embodiments, a complex (e.g., two or more) minerals are used as a colloidal mineral complex. The colloidal mineral complex can include any suitable mineral compounds and/or their salts. In some embodiments, the colloidal mineral complex includes, without limitation, one or more of calcium, magnesium, inorganic phosphate, citrate, sodium, potassium, and chloride, or their respective salts. In some embodiments, the colloidal mineral complex is present in an amount between about 2% and about 15%, e.g., about 2% to about 12%, about 5% to about 10%, about 5% to about 9%, including about 6% to about 9% (by weight) of the phosphoprotein mass in the composition. In some embodiments, the colloidal mineral complex is present in an amount of about 2%, 3%, 4%, 5%, 6%, 7%, 8%, 9%, 10%, 11%, 12%, 13%, 14%, 15% or an amount within a range defined by any two of the preceding percentages.

The present composition generally includes a physiologically compatible excipient, such as but not limited to water or a buffer. Generally, the physiologically compatible excipient is an excipient that does not substantially interfere with the protective properties of the casein phosphoproteins (e.g., does not substantially interfere with the micellar structures formed by the casein phosphoproteins and/or their protective properties). Suitable physiologically compatible excipients include, but are not limited to, saline, aqueous buffer solutions, solvents and/or dispersion media. In some embodiments, the physiologically compatible excipient is phosphate buffered saline (PBS). Other non-limiting examples of materials which can serve as physiologically compatible excipients include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. In some embodiments, the physiologically compatible excipient is a beverage. In some embodiments, the physiologically compatible excipient is a liquid infant formula. In some embodiments, the excipient inhibits the degradation of the active agent, e.g., the nucleic acid containing micelles.

In some embodiments, the composition contains casein micelles that include the following (by weight): about 30-40% (e.g., about 36%) alpha S1 casein, about 5-15% (e.g., about 10%) alpha s2 casein, about 30-40% (e.g., about 34%) beta casein, about 5-15% (e.g., about 12%) kappa casein, and about 7% colloidal mineral complex (including phosphate, calcium, magnesium and citrate) in phosphate-buffered saline, where the total amount of casein phosphoproteins is about 8% of the composition, weight by volume.

Also provided are methods of preparing a composition for enhanced oral bioavailability of therapeutic nucleic acids, as described herein. The method in general includes combining: a therapeutic nucleic acid, at least two phosphoproteins selected from alpha s1 casein, alpha s2 casein, beta casein, and kappa casein; and a physiologically compatible excipient, under conditions sufficient to form micelles comprising the at least two phosphoproteins in the composition. The components of the composition may be combined using any suitable option. In some embodiments, the casein phosphoproteins are first combined with the physiologically compatible excipient to make a casein composition, and then, the nucleic acid is combined with the casein composition to generate the composition for enhanced oral bioavailability of the nucleic acid. Any suitable option may be used to generate the casein composition. Suitable methods of providing a composition with casein phosphoproteins are described, e.g., in European Pat. No. 2732710B1, the entire disclosure of which is incorporated herein by reference.

In some embodiments, one or more components of the composition are preserved and can be reconstituted into a composition for orally administering the nucleic acid to a subject. In some embodiments, the casein phosphoproteins of the composition are preserved, and are reconstituted into the composition for orally administering the nucleic acid to a subject. In some embodiments, the nucleic acids are preserved, and are reconstituted into a composition (e.g., a composition with casein phosphoproteins and a physiologically compatible excipient) for orally administering to a subject. “Preserved” as used herein, can describe a state in which the functional activity of the nucleic acids (whether alone, or integrated into the compositions provided for herein), as described herein, is retained for at least a defined period under standard storage conditions. In some embodiments, the preserved nucleic acid retains 10% or more, e.g., 20% or more, 30% or more, 40% or more, 50% or more, 60% or more, 70% or more, 75% or more, 80% or more, 85% or more, 90% or more, 95% or more, or about 100% of the functional activity when reconstituted after storage compared to the functional activity before being preserved.

In some embodiments, the nucleic acids (e.g., encapsulated in casein-chitosan coated micelles) are preserved and stored under standard storage conditions for 1 day or more, e.g., 2 days or more, 5 days or more, 2 weeks or more, one month or more, 3 months or more, 6 months or more, 1 year or more, 3 years or more, 5 years or more, including 10 years or more. In some embodiments, the nucleic acids (e.g., encapsulated in casein-chitosan coated micelles) are preserved and stored under standard storage conditions for a period of 1 day to 5 years, e.g., 5 days to 3 years, 10 days to 2 years, one month to 1 year, including 3 months to 6 months.

In some embodiments, the nucleic acids (e.g., encapsulated in casein-chitosan coated micelles) are stored under any suitable standard storage conditions. In some embodiments, a standard storage condition includes a temperature of 25° C. or lower, e.g., 20° C. or lower, 15° C. or lower, 10° C. or lower, 5° C. or lower, 0° C. or lower, −10° C. or lower, −20° C. or lower, −30° C. or lower, −40° C. or lower, −50° C. or lower, −60° C. or lower, −70° C. or lower, including −80° C. or lower. In some embodiments, a standard storage condition has a temperature in the range of −90° C. to −80° C., −80° C. to −70° C., −70° C. to −60° C., −60° C. to −50° C., −50° C. to −40° C., −40° C. to, −30° C., −30° C. to −20° C., −20° C. to −10° C., −10° C. to −5° C., −5° C. to 0° C. 0° C. to 5° C., 5° C. to 10° C. 10° C. to 15° C., 15° C. to 20° C., 20° C. to 25° C., 25° C. to 30° C., or 30° C. to 35° C. In some embodiments, a standard storage condition is at room temperature and standard atmospheric pressure.

In some embodiments, the nucleic acids (e.g., encapsulated in casein-chitosan coated micelles) are preserved in any suitable manner. In some embodiments, they are frozen. Means for freezing exosomes are described in, e.g., Bosch et al., Sci Rep. 2016 November 8; 6:36162. In some embodiments, they are lyophilized. Means for lyophilizing exosomes are described in, e.g., PCT Publication No. WO2018070939.

In some embodiments, the nucleic acids (e.g., encapsulated in casein-chitosan coated micelles) are reconstituted into a composition using any suitable options. In some embodiments, the exosomes are reconstituted into a physiologically acceptable excipient, such as water or a buffer solution.

The compositions of the present disclosure find use in a variety of situations where systemic delivery of therapeutic nucleic acids (e.g., coding or non-coding RNAs) is desired, e.g., to treat a disorder or disease that can be treated by systemic delivery of therapeutic nucleic acids. Thus, provided herein are methods that include orally administering to a subject any of the compositions as described herein. In some embodiments, the subject has or is at risk of developing a myodegenerative disorder, and the composition comprises therapeutic nucleic acids, to thereby treat the myodegenerative disorder. In some embodiments, the subject is human.

The compositions of the present disclosure can provide for enhanced bioavailability of the therapeutic nucleic acids when the composition is administered orally. In some embodiments, the bioavailability is enhanced compared to a suitable control composition, e.g., a composition that does not include the casein phosphoproteins, the chitosan polymers, and/or the casein-chitosan complex. In some embodiments, the oral bioavailability of the therapeutic nucleic acids in the composition is enhanced by about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 25 fold, about 30 fold, about 40 fold, about 50 fold, about 60 fold, about 70 fold, about 80 fold, about 90 fold, about 100 fold, about 200 fold, about 300 fold, about 400 fold, about 500 fold, about 1,000 fold, about 2,000 fold, about 5,000 fold, about 10,000 fold or more, or enhanced by a fold amount within a range defined by any two of the preceding values, compared to the oral bioavailability of a control composition

In some embodiments, the enhanced oral bioavailability includes increased representation of different species within a class of payload molecules (e.g., unique nucleic acid sequences among all nucleic acid sequences). In some embodiments, substantially all species within a class of payload molecules are bioavailable. In some embodiments, at least about 50%, e.g., at least about 60%, at least about 70%, at least about 75%, at least about 80%, at least about 85%, at least about 90%, at least about 95%, at least about 97%, at least about 98%, including at least about 99% of species within a class of payload molecules are bioavailable. In some embodiments, the payload molecules are exosomal nucleic acids. In some embodiments, the payload molecules are mRNA, ncRNA, lncRNA, or miRNA. In some embodiments, the payload molecules are proteins. In some embodiments, the payload molecules are small molecules. In some embodiments payload molecules are synthetic payloads, e.g., synthetic nucleic acids, proteins, or small molecules.

In some embodiments, the enhanced oral bioavailability is achieved by the composition within about 1 week, within about 5 days, within about 3 days, within about 1 day, within about 18 hours, within about 12 hours, within about 10 hours, within about 8 hours, within about 6 hours, within about 5 hours, within about 4 hours, within about 3 hours, within about 2 hours, within about 1 hour, within about 30 minutes, within about 15 minutes, or within any interval of time in a range defined by any two of the preceding values after oral administration.

In several embodiments, the chitosan is low molecular weight chitosan. In several embodiments, the chitosan ranges from about ranges in mass from about 50 to about 190 kiloDaltons. In some embodiments, higher molecular weight chitosan is used. In several embodiments, the chitosan is present in an amount ranging from about 0.001 to about 0.1% of the formulation by weight per volume. In several embodiments, the chitosan is present in an amount ranging from about 0.01 to about 0.1% of the formulation by weight per volume. In several embodiments, the chitosan is present in an amount ranging from about 0.05 to about 0.1% of the formulation by weight per volume.

In several embodiments, the formulation is generated based on ratios of the various components of the formulation. For example, is the ratio of casein to RNA to chitosan ranges from about 1000:1:25 to about 500:1:15 (% w/v). In several embodiments, the ratio of casein to RNA ranges from about 500:1 to about 1000:1. In several embodiments, the ratio of RNA to chitosan ranges from about 1:10 to about 1:50. In several embodiments, these ratios are employed with a volume of liposome that ranges from about 0.5 to about 3 microliters for each microgram of RNA used. Ratios may also be used between any two given components of a composition, ranging from 1:1, 2:1, 3:1, 4:1, 5:1, 10:1, 50:1, 100:1, 1000:1, 1:1000, 1:500, 1:100, 1:50, 1:5, 1:4, 1:3, 1:2, etc.

In some alternative embodiments, the composition includes extracellular vesicles (EV), e.g., exosomes. The extracellular vesicles (EV) can be those from any suitable source, e.g., EV derived from cardiosphere-derived cells (CDC), or from fibroblasts. Suitable EV, such as CDC-derived EV, are provided in, e.g., U.S. Application Publication Nos. 20080267921, 20160158291 and 20160160181; Smith et al., Circulation. 2007. 115:896-908; Aminzadch, M. A. et al. Stem Cell Reports 10, 942-955 (2018); and Ibrahim et al., Stem Cell Reports. 2014 May 8; 2(5):606-19, Ibrahim, A. G. et al. Nanomedicine 33, 102347 (2020), each of which is incorporated by reference in its entirety. In some embodiments, the EVs are those isolated from serum-free media conditioned by human CDCs in culture. In some embodiments, the composition includes EV and liposomes and/or PCLC as transfection reagents. In some embodiments, the composition is substantially free of CDC-derived EV.

EVs, e.g., exosomes, disclosed herein can vary in size, depending on the embodiment. Depending on the embodiment, the size of the EVs ranges in diameter from about 15 nm to about 95 nm in diameter, including about 15 nm to about 20 nm, about 20 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 95 nm, and overlapping ranges thereof. In several embodiments, EVs are larger (e.g., those ranging from about 140 to about 210 nm, including about 140 nm to about 150 nm, about 150 nm to about 160 run, about 160 nm to about 170 nm, about 170 nm to about 180 nm, about 180 nm to about 190 nm, 190 nm to about 200 nm, about 200 nm to about 210 nm, and overlapping ranges thereof). In some embodiments, the EV diameter is in a range of about 15 nm to about 200 nm in diameter, including about 15 nm to about 20 nm, about 20 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 100 nm, about 100 nm to about 110 nm, about 110 nm to about 120 nm, about 120 nm to about 130 nm, about 130 nm to about 140 nm, about 140 nm to about 150 nm, about 150 nm to about 160 nm, about 160 nm to about 170 nm, about 170 nm to about 180 nm, about 180 nm to about 190 nm, about 190 nm to about 200 nm, and overlapping ranges thereof. In some embodiments, the EVs that are generated from the original cellular body are 100, 200, 300, 400, 500, 600, 700, 800, 900, 1,000, 2,000, 5,000, or 10,000 times smaller in at least one dimension (e.g., diameter) than the original cellular body.

The composition containing the EV and nucleic acid of the present disclosure can be prepared using any suitable option. In some embodiments, loading the nucleic acid into the EV includes: formulating the nucleic acid with liposomes and/or PCLC. e.g., as provided above, to generate a nucleic acid-liposome mixture; combining the nucleic acid-liposome mixture with the EV; and enriching for EV associated with exosome markers to generate a population of EV enriched for the nucleic acid. Combining the nucleic acid-liposome mixture with the EV can be done using any suitable option. In some embodiments, the nucleic acid-liposome mixture is combined with the EV at 37° C. with shaking for about 30 minutes or more. Enriching to generate a population of EV enriched for the nucleic acid can be done using any suitable option. In some embodiments, enriching for EV associated with exosome markers includes immunoprecipitating EV associated with exosome markers using antibodies specific to an exosome marker. In some embodiments, the exosome marker is one or more of CD9, CD63 and CD81. In some embodiments, enriching for EV associated with exosome markers includes immunoprecipitating EV associated with all the exosome markers, CD9, CD63 and CD81. In some embodiments, the size distribution of the population of EV enriched for the nucleic acid is substantially unimodal. In some embodiments, at least 80%, 85%, 90%, 95%, 97%, 99% of the population has a diameter under a single peak in the size distribution. In some embodiments, the population of EV enriched for the nucleic acid has an average diameter of about 50-180 nm, e.g., 60-170 nm, 70-160 nm, 80-150 nm, 90-140 nm, 100-130 nm, or about 110-130 nm.

### Nucleic Acids

As discussed above, nucleic acid therapeutics offer the potential to treat diseases at a genetic level. Because conventional treatments generally target proteins, rather than the underlying genetic cause, the induced therapeutic effects may be transient. In contrast, nucleic acid therapeutics have the potential for long-lasting (or even permanent, e.g., curative) effects via gene inhibition, addition, replacement, or editing. As disclosed herein, the successful use of nucleic acid therapeutics will hinge on delivery technologies that improve stability and/or bioavailability.

In several embodiments, the formulations provided for herein allow the enhanced delivery of nucleic acids to a subject. In several embodiments, the nucleic acids comprise DNA. In several embodiments, the nucleic acids comprise RNA. In several embodiments, the nucleic acids comprise coding RNA (e.g., messenger RNA or mRNA). In several embodiments, the nucleic acids comprise non-coding RNAs (ncRNA). mRNA can provide therapeutic effects by virtue of delivery of a sequence coding for a protein that yields a therapeutic effect. Depending on the embodiment, that could be a protein that replaces a non-functional protein. In other embodiments, the coding RNA could encode a protein to which an immune response is desired (e.g., a viral protein), such as for the production of antibodies against that protein. ncRNA are known to exhibit positive therapeutic effects based on their ability to increase secretion of anti-inflammatory cytokines or decrease secretion of inflammatory cytokines. Certain ncRNA exhibit cardioprotective, anti-fibrotic, and/or anti-hypertrophic effects.

Non-coding RNAs (ncRNAs) are increasingly recognized as bioactive. Extracellular vesicles (EVs) derived from progenitor cells contain plentiful and diverse ncRNAs; cardiosphere-derived cells, for example, secrete EVs rich in small Y RNAs. Nucleic acids that exhibit therapeutic effects can be delivered using the formulations provided for herein. By way of non-limiting example, ncRNAs that suppress hypertrophic, inflammatory and fibrotic gene families in isolated macrophages, restore cardiac function and exercise endurance, and/or reduce serum biomarkers of heart failure and inflammation can be used according to embodiments disclosed herein. Additionally, ncRNA that antagonize upregulation of ERK/Map Kinase, fibrotic and inflammatory signaling in tissues can be used. Any ncRNA that exhibits positive effects against pathological processes as diverse as ischemia (myocardial infarction), myodegeneration (Duchenne muscular dystrophy) and autoimmunity (scleroderma) or exhibit other positive disease-modifying bioactivity can be used according to embodiments disclosed herein. Table 1 includes non-limiting example of such ncRNA to use as a therapeutic to treat diseases associated with inflammation and/or fibrosis.

TABLE 1

| | | SEQ |
|---|---|---|
| | | ID |
| Name | Sequence | NO: |
| EV-YF1 variant | CUGGUCCGAUGGUAGUGGGUUA | 7 |
| 2from5 | UCAGAACUUAUUAACAUUAGUG | |
| | UCACUAAAGU | |
| EV-YF1 variant | CUGGUCCGAUGGUAGUGGGUUA | 8 |
| 2from5_54U to A | UCAGAACUUAUUAACAUUAGUG | |
| | UCACUAAAGA | |
| EV-YF1 variant | CUGGUCCGAUGGUAGUGGGUUA | 9 |
| 2from5_54U to A | UCAGAACUUAUUAACAUUAGUG | |
| LNA_Gapmer | UCACUAAAGA | |
| EV-YF1 variant | CUGGUCCGAUGGUAGUGGGUUA | 10 |
| 2from5_54U to A | UCAGAACUUAUUAACAUUAGUG | |
| LNA_Mixmer | UCACUAAAGA | |
| TY4 (Native) | GGUCCGAUGGUAGUGGGUUAUC | 11 |
| | AG | |
| TY4 1G to C | CGUCCGAUGGUAGUGGGUUAUC | 12 |
| | AG | |
| TY4 1G to C | CGUCCGAUGGUAGUGGGUUAUC | 13 |
| LNA_Gapmer | AG | |
| TY4 1G to C | CGUCCGAUGGUAGUGGGUUAUC | 2 |
| LNA Mixmer | AG | |
| EV-YF1 | GGCUGGUCCGAUGGUAGUGGGU | 1 |
| | UAUCAGAACUUAUUAACAUUAG | |
| | UGUCACUAAAGU | |
| Fomivirsen | GCGTTTGCTCTTCTTCTTGCG | 14 |
| Mipomersen | GCCUCAGTCTGCTTCGCACC | 15 |
| Nusinersen | UCACUUUCAUAAUGCUGG | 16 |
| Eteplirsen | CTCCAACATCAAGGAAGATG | 17 |
| | GCATTTCTAG | |
| Inotersen | TCTTGGTTACATGAAATCCC | 18 |
| Patisiran | GUAACCAAGAGUAUUCCAU | 19 |
| | TT | |
| Golodirsen | GTTGCCTCCGGTTCTGAAGG | 20 |
| | TGTTC | |
| Givosiran | CAGAAAGAGUGUCUCAUCU | 21 |
| | UA | |
| Viltolarsen | CCTCCGGTTCTGAAGGTGT | 22 |
| | TC | |
| Volanesorsen | AGCTTCTTGTCCAGCTTT | 23 |
| | AT | |
| Inclisiran | CUAGACCUGUTUUGCUUUU | 24 |
| | GU | |
| Lumasiran | GACUUUCAUCCUGGAAAUA | 25 |
| | UA | |
| piR-659/piREX1 | CCCCCCACUGCUAAAUUUG | 31 |
| | ACUGGUU | |
| piR-659/piREX1 | CCCCCCACUGCUAAAUUUG | 32 |
| U to A | ACUGGUA | |

Provided herein is an isolated nucleic acid that includes a nucleotide sequence of CGUCCGAUGGUAGUGGGUUAUCAG (SEQ ID NO: 12), or a variant thereof. In some embodiments, the nucleic acid is RNA. In some embodiments, the nucleic acid includes a nucleotide sequence at least 80%, 85%, 90%, 92%, 93%, 94%, 95%, 96%, 98%, 99% identical to CGUCCGAUGGUAGUGGGUUAUCAG (SEQ ID NO: 12), or to any of the nucleic acid sequences provided for herein. In some embodiments, the nucleic acid includes a nucleotide sequence of CGUCCGAUGGUAGUGGGUUAUCAG (SEQ ID NO: 12) with a sequence variation at up to 1, 2, 3, 4, or 5 positions in the nucleotide sequence. As used herein, a “position” within a nucleotide sequence or nucleic acid is defined relative to the 5′ end of the nucleotide sequence or nucleic acid. In some embodiments, the nucleotide sequence of the nucleic acid is CGUCCGAUGGUAGUGGGUUAUCAG (SEQ ID NO: 12), or a sequence variant thereof. The nucleic acid can be any suitable length. In some embodiments, the nucleic acid is 24 nucleotides (nt) long. In some embodiments, the nucleic acid is 16, 17, 18, 19, 20, 21, 22, 23, 24, 25, 26, 27, 28, 29, 30, 31, 32, 33, 34, 35, 36, 37, 38, 39, 40 nt long, or longer. In some embodiments, the nucleic acid is at most 30 nt long. In some embodiments, the nucleic acid is 16-30 nt long, or 24-30 nt long.

A nucleic acid of the present disclosure can be single stranded or double stranded (e.g., RNA/DNA hybrid). In some embodiments, the nucleic acid is single stranded.

An isolated nucleic acid of the present disclosure in some embodiments includes one or more chemically-modified nucleotides, e.g., nucleotides with a modified backbone. In general, the chemical modification(s) is one that substantially preserves or enhances the therapeutic potency of the nucleic acid. Any suitable number of nucleotides of the nucleic acid can be chemically modified. In some embodiments, the nucleic acid includes 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more chemically-modified nucleotides. In some embodiments, the nucleic acid includes 1-5, 1-6, 1-7, 1-8, 1-9, 1-10, 1-15, 1-20, 1-25, or 1-30 chemically-modified nucleotides. In some embodiments, the nucleic acid includes 1-10 chemically-modified nucleotides. In some embodiments, the nucleic acid includes 8 chemically-modified nucleotides. In some embodiments, the nucleic acid includes 6 chemically-modified nucleotides.

The chemically modified nucleotides can be distributed along the isolated nucleic acid in any suitable manner. In some embodiments, the nucleic acid includes at least one chemically-modified nucleotide within the first half of the nucleic acid, e.g., the 5′ half of the nucleic acid. In some embodiments, the nucleic acid includes at least one chemically-modified nucleotide within the second half of the nucleic acid, e.g., the 3′ half of the nucleic acid. In some embodiments, the nucleic acid includes at least one chemically-modified nucleotide within the first half of the nucleic acid, e.g., the 5′ half of the nucleic acid, and at least one chemically-modified nucleotide within the second half of the nucleic acid, e.g., the 3′ half of the nucleic acid. In some embodiments, the nucleic acid includes one or more chemically-modified nucleotides within 1, 2, 3, 4, 5, 6, 7, 8, 9, 10 or more nucleotides from the 5′ end of the nucleic acid. In some embodiments, the nucleic acid includes one or more chemically-modified nucleotides within 1, 2, 3, 4, 5, 6, 7, 8, 9, 10 or more nucleotides from the 3′ end of the nucleic acid. In some embodiments, no two chemically-modified nucleotides are adjacent each other in the nucleic acid. In some embodiments, the nucleic acid includes 1, 1, 2, 2, 3, 3, 4, 4, 5, 5 chemically-modified nucleotides within 1, 2, 3, 4, 5, 6, 7, 8, 9, 10 nucleotides, respectively, from the 5′ end of the nucleic acid. In some embodiments, the nucleic acid includes 1, 1, 2, 2, 3, 3, 4, 4, 5, 5 chemically-modified nucleotides within 1, 2, 3, 4, 5, 6, 7, 8, 9, 10 nucleotides, respectively, from the 3′ end of the nucleic acid. In some embodiments, the nucleic acid includes the same number of chemically-modified nucleotides in the 5′ half and 3′ half of the nucleic acid. In some embodiments, the nucleic acid includes 3 chemically-modified nucleotides within 5 nucleotides from the 5′ end of the nucleic acid and/or 3 chemically-modified nucleotides within 5 nucleotides from the 3′ end of the nucleic acid.

In some embodiments, the chemically-modified nucleotides are within the nucleotide sequence of CGUCCGAUGGUAGUGGGUUAUCAG (SEQ ID NO: 12), or sequence variant thereof, or other nucleic acid sequence provided for herein. In some embodiments, the nucleic acid includes at least one chemically-modified nucleotide within the first half of the nucleotide sequence, e.g., the 5′ half of the nucleotide sequence. In some embodiments, the nucleic acid includes at least one chemically-modified nucleotide within positions 1-12 of the nucleotide sequence. In some embodiments, the nucleic acid includes at least one chemically-modified nucleotide within the second half of the nucleotide sequence, e.g., the 3′ half of the nucleotide sequence. In some embodiments, the nucleic acid includes at least one chemically-modified nucleotide within positions 13-24 of the nucleotide sequence. In some embodiments, the nucleic acid includes at least one chemically-modified nucleotide within positions 1-12 of the nucleotide sequence, and at least one chemically-modified nucleotide within positions 13-24 of the nucleotide sequence. In some embodiments, the nucleic acid includes one or more chemically-modified nucleotides within 1, 2, 3, 4, 5, 6, 7, 8, 9, 10 or more nucleotides from the 5′ end of the nucleotide sequence. In some embodiments, the nucleic acid includes one or more chemically-modified nucleotides within 1, 2, 3, 4, 5, 6, 7, 8, 9, 10 or more nucleotides from the 3′ end of the nucleotide sequence. In some embodiments, no two chemically-modified nucleotides are adjacent each other in the nucleotide sequence. In some embodiments, the nucleic acid includes 1, 1, 2, 2, 3, 3, 4, 4, 5, 5 chemically-modified nucleotides within 1, 2, 3, 4, 5, 6, 7, 8, 9, 10 nucleotides, respectively, from the 5′ end of the nucleotide sequence. In some embodiments, the nucleic acid includes 1, 1, 2, 2, 3, 3, 4, 4, 5, 5 chemically-modified nucleotides within 1, 2, 3, 4, 5, 6, 7, 8, 9, 10 nucleotides, respectively, from the 3′ end of the nucleotide sequence. In some embodiments, the nucleic acid includes the same number of chemically-modified nucleotides in the 5′ half and 3′ half of the nucleotide sequence. In some embodiments, the nucleic acid includes 3 chemically-modified nucleotides within 5 nucleotides from the 5′ end of the nucleotide sequence and/or 3 chemically-modified nucleotides within 5 nucleotides from the 3′ end of the nucleotide sequence. In some embodiments, the nucleic acid includes a different number of chemically-modified nucleotides in the 5′ half and 3′ half of the nucleotide sequence. In some embodiments, the nucleic acid includes a greater number of chemically-modified nucleotides in the 3′ half than in the 5′ half of the nucleotide sequence. In some embodiments, the nucleic acid includes 3 chemically-modified nucleotides within 5 nucleotides from the 5′ end of the nucleotide sequence and/or 3, 4, or 5 chemically-modified nucleotides within 5 nucleotides from the 3′ end of the nucleotide sequence.

In some embodiments, the isolated nucleic acid includes a chemically-modified nucleotide at one or more of positions 1, 3, 5, 20, 22 and 24 of the nucleotide sequence. In some embodiments, the isolated nucleic acid includes a chemically-modified nucleotide at positions 1, 3, 5, 20, 22 and 24 of the nucleotide sequence. In some embodiments, the isolated has nucleic acid the nucleotide sequence CGUCCGAUGGUAGUGGGUUAUCAG (SEQ ID NO: 12), or a sequence variant thereof, where one or more of positions 1, 3, 5, 20, 22, and 24 are chemically modified. In some embodiments, the chemically-modified nucleotide(s) increases in vitro and/or in vivo stability of the nucleic acid. In some embodiments, the chemically-modified nucleotide(s) increases therapeutic potency of the nucleic acid, e.g., for treating an inflammatory condition, cardiac injury, or muscular dystrophy.

The isolated nucleic acid in some embodiments includes one type, or two or more different types of chemically-modified nucleotides. In some embodiments, the chemically-modified nucleotide has a methylene bridge connecting the 2′-O atom and the 4′-C atom of the nucleotide sugar ring to lock the conformation (Locked Nucleic Acid (LNA)). In some embodiments, the isolated nucleic acid includes the nucleotide sequence CGUCCGAUGGUAGUGGGUUAUCAG (SEQ ID NO: 12), or a sequence variant thereof, where one or more of positions 1, 3, 5, 20, 22, and 24 are LNA. In some embodiments, the isolated nucleic acid has the nucleotide sequence CGUCCGAUGGUAGUGGGUUAUCAG (SEQ ID NO: 12), or a sequence variant thereof, where one or more of positions 1, 3, 5, 20, 22, and 24 are LNA. In some embodiments, the isolated nucleic acid includes the nucleotide sequence CGUCCGAUGGUAGUGGGUUAUCAG (SEQ ID NO: 2), or a sequence variant thereof, where positions 1, 3, 5, 20, 22, and 24 are LNA. In some embodiments, the isolated nucleic acid has the nucleotide sequence CGUCCGAUGGUAGUGGGUUAUCAG (SEQ ID NO: 2), where positions 1, 3, 5, 20, 22, and 24 are LNA.

The isolated nucleic acid, in some embodiments, can include any suitable chemical modification. In some embodiments, the chemical modification is a backbone modification, e.g., modification of the sugar/phosphate backbone. In some embodiments, the chemical modification is a backbone sugar modification. In some embodiments, the chemically modified nucleotide includes a LNA. In some embodiments, the chemical modification includes the introduction of a phosphorothioate group as linker between nucleotides. Suitable backbone modifications of the chemically-modified nucleotides include, without limitation, phosphorothioates, phosphotriesters, methyl phosphonates, short chain alkyl or cycloalkyl intersugar linkages or short chain heteroatomic or heterocyclic intersugar linkages. In some embodiments, the chemical modification is a base modification.

The nucleic acids of the present disclosure can be prepared using any suitable option. Suitable options include, without limitation, chemical synthesis, enzymatic production and/or biological production. In some embodiments, the nucleic acids are prepare using chemical synthesis. Any suitable option for chemical synthesis of nucleic acids can be used. Suitable options include, without limitation, phosphodiester, phosphotriester, phosphoramidite, phosphite-triester, and solid phase synthesis approaches. I In some embodiments, preparing the nucleic acids includes in vitro transcription. In some embodiments, the nucleic acids are prepared using recombinant DNA technology. In some embodiments, the nucleic acids are prepared by chemically modifying an unmodified nucleic acid having a nucleotide sequence of interest.

### Methods

Provided herein are methods of treating a subject in need thereof using the formulations of the present disclosure (also referred to herein as “treatment methods”) which comprise oral formulations that carry nucleic acids (e.g., RNA) as a therapeutic payload. Conditions that may be treated by the treatment methods include, but are not limited to, those diseases associated with, for example, inflammation and/or fibrosis. Conditions include, without limitation, heart conditions, muscular disorders, myocardial infarction, cardiac disorders, myocardial alterations, muscular dystrophy, fibrotic disease, inflammatory disease, viral infection, scleroderma, heart failure with preserved ejection fraction, sepsis and/or wound healing. In some embodiments, the conditions treated by the present treatment methods are a symptom and/or sequelae of an infection. In some embodiments, the infection is a viral infection, e.g., a respiratory virus infection, such as COVID-19, infections due to other coronaviruses, or other viral pathogens (e.g., flu, H1N1, Hepatitis C, HIV, etc.).

In some embodiments, a treatment method includes a method of treating a muscle disorder (or muscle condition) or symptom thereof, the method including orally administering to a subject in need of treating a muscle disorder or symptom thereof a therapeutically effective amount of one or more of the compositions containing a nucleic acid of the present disclosure. The muscle disorder can be, without limitation, a skeletal muscle disorder or a cardiac muscle disorder. In some embodiments, the muscle disorder includes muscular dystrophy, e.g., Duchenne muscular dystrophy. In some embodiments, the subject has muscular dystrophy, or is at risk of developing muscular dystrophy. In some embodiments, the subject is genetically predisposed to developing muscular dystrophy, e.g., Duchenne muscular dystrophy. In some embodiments, the subject has one or more mutations in a dystrophin gene that predisposes the subject to developing muscular dystrophy, e.g., Duchenne muscular dystrophy.

In some embodiments, a treatment method includes a method of treating a heart condition or symptom thereof, the method including orally administering to a subject in need of treating a heart condition or symptom thereof a therapeutically effective amount of the one or more of the compositions containing a nucleic acid of the present disclosure. In some embodiments, the subject is a human subject. In some embodiments, the subject is a non-human subject, e.g., a non-human mammal.

A variety of heart conditions may be treated by the present method. In some embodiments, the heart condition includes a symptom and/or sequelae of heart failure or myocardial infarction. In some embodiments, the heart condition includes hypertrophic cardiomyopathy. In some embodiments, the heart condition includes heart failure with preserved ejection fraction (HFpEF).

In some embodiments, the subject is at risk of developing the heart condition. In some embodiments, the subject is at risk of developing the heart condition based on one or more of the subject's family history, genetic predisposition, life style, and medical history. In some embodiments, the subject has a mutation in cardiac troponin I that predisposes the subject to developing hypertrophic cardiomyopathy (HCM). In some embodiments, the subject has one or more comorbidities for the heart condition. In some embodiments, the one or more comorbidities includes obesity and hypertension. In some embodiments, the subject has, or is diagnosed with, the heart condition.

In some embodiments, the subject exhibits one or more of: hypertension, elevated E/e′ ratio, cardiac hypertrophy, myocardial fibrosis, obesity, wasting, reduced endurance, and elevated systemic inflammatory markers. In some embodiment, the subject has hypertension, and administering the therapeutically effective amount of the nucleic acid (or composition thereof) reduces the subject's blood pressure. In some embodiments, a subject having hypertension has a resting blood pressure of over 130/90 mmHg. In some embodiments, a subject having hypertension has a resting blood pressure of over 140/90 mmHg. In some embodiment, administering the therapeutically effective amount of the nucleic acid (or composition thereof) reduces the subject's systolic blood pressure or diastolic blood pressure. In some embodiment, the subject's blood pressure (systolic or diastolic blood pressure) is reduced by at least about 5%, 7.5%, 10%, 12.5%, 15%, 17.5%, 20%, 25%, 30% or more, or by a percentage in a range defined by any two of the preceding values, after administering the therapeutically effective amount of the nucleic acid (or composition thereof). In some embodiment, the subject's blood pressure (systolic or diastolic blood pressure) is reduced at least to a level that is deemed no longer to be hypertensive after administering the therapeutically effective amount of the nucleic acid (or composition thereof).

In some embodiment, the subject has an elevated E/e′ ratio, and administering the therapeutically effective amount of the nucleic acid (or composition thereof) reduces the E/e′ ratio. In some embodiment, the subject's E/e′ ratio is reduced by at least about 5%, 7.5%, 10%, 12.5%, 15%, 17.5%, 20%, 25%, 30%, 35%, 40%, 45%, 50%, 55%, 60%, 65%, 70%, 75% or more, or by a percentage in a range defined by any two of the preceding values, after administering the therapeutically effective amount of the nucleic acid (or composition thereof). In some embodiment, the subject's E/e′ ratio is reduced at least to a level that is deemed no longer to be clinically relevant after administering the therapeutically effective amount of the nucleic acid (or composition thereof).

In some embodiment, the subject has cardiac hypertrophy, and oral administration of the therapeutically effective amount of a composition comprising a nucleic acid reduces cardiac hypertrophy. Cardiac hypertrophy can be measured using any suitable option. In some embodiments, cardiac hypertrophy is measured using echocardiography. In some embodiments, a subject having cardiac hypertrophy has an increased diastolic interventricular septal wall diameter (IVSd) and/or left ventricular posterior wall diameter (LVPWd), as measured by echocardiography, and administering the therapeutically effective amount of the nucleic acid (or composition thereof) reduces the IVSd and/or LVPWd. In some embodiment, the subject's IVSd or LVPWd is reduced by at least about 5%, 7.5%, 10%, 12.5%, 15%, 17.5%, 20%, 25%, 30% or more, or by a percentage in a range defined by any two of the preceding values, after administering the therapeutically effective amount of the nucleic acid (or composition thereof). In some embodiment, the subject's IVSd or LVPWd is reduced at least to a level that is deemed no longer to be hypertrophic after administering the therapeutically effective amount of the composition comprising a nucleic acid.

In some embodiment, the subject has myocardial fibrosis, and orally administering the therapeutically effective amount of a composition comprising a therapeutic nucleic acid as provided for herein prevents or reduced fibrosis. “Fibrosis” as used herein can include any remodeling (e.g., pathological remodeling) of the myocardium, such as, but not limited to, deposition of fibrotic and/or fatty tissue, replacement of muscle tissue with fibrotic and/or fatty tissue, etc. Cardiac fibrosis is monitored using any suitable means, such as biopsy, ultrasonography or MRI. In some embodiments, orally administering a composition comprising a therapeutic nucleic acid as provided for herein eliminates or retards the development of myocardial fibrosis and/or muscle fibrosis.

In some embodiments, the subject exhibits wasting or weight loss, and orally administering a composition comprising a therapeutic nucleic acid as provided for herein retards or prevents the wasting. In some embodiment, the subject exhibits body weight loss of at most about 20%, 15%, 10%, 5%, 3% or less, or a percentage in a range defined by any two of the preceding values, after administering the therapeutically effective amount of the nucleic acid (or composition thereof). In some embodiment, the subject's body weight recovers to, or is maintained at substantially the pre-treatment level after orally administering a composition comprising a therapeutic nucleic acid as provided for herein.

In some embodiments, the subject exhibits reduced endurance, e.g., exercise endurance, and orally administering a composition comprising a therapeutic nucleic acid as provided for herein retards or prevents the decline in endurance. In some embodiment, the subject exhibits a decline in endurance of at most about 20%, 15%, 10%, 5%, 3% or less, or a percentage in a range defined by any two of the preceding values, after orally administering a composition comprising a therapeutic nucleic acid as provided for herein. In some embodiments, the subject's exercise endurance recovers to, or is maintained at substantially the pre-treatment level after orally administering a composition comprising a therapeutic nucleic acid as provided for herein. In some embodiment, the subject exhibits an improvement in endurance of at least about 5%, 10%, 15%, 20%, 25%, 30%35%, 40%, 50% or more, or a percentage in a range defined by any two of the preceding values, after orally administering a composition comprising a therapeutic nucleic acid as provided for herein. In some embodiments, the improvement in endurance after orally administering a composition comprising a therapeutic nucleic acid as provided for herein is sustained over the duration of treatment. In some embodiments, the improvement in endurance after orally administering a composition comprising a therapeutic nucleic acid as provided for herein is sustained across multiple doses of administration.

In some embodiments, the subject exhibits elevated levels of systemic inflammatory markers, e.g., in the peripheral blood. In some embodiments, the systemic inflammatory marker includes one or more of IL-6 and brain natriuretic peptide (BNP). In some embodiments, the level of the systemic inflammatory marker is reduced by at least about 5%, 7.5%, 10%, 12.5%, 15%, 17.5%, 20%, 25%, 30%, 35%, 40%, 45%, 50%, 55%, 60%, 65%, 70%, 75% or more, or by a percentage in a range defined by any two of the preceding values, after orally administering a composition comprising a therapeutic nucleic acid as provided for herein. In some embodiment, the subject's systemic inflammatory marker is reduced at least to a level that is deemed no longer to be elevated after orally administering a composition comprising a therapeutic nucleic acid as provided for herein.

In some embodiments, where the subject is obese, the therapeutic effect of administering the nucleic acid is independent of the subject's obesity. In some embodiments, orally administering a composition comprising a therapeutic nucleic acid as provided for herein does not affect the subject's weight.

In some embodiments, the subject exhibits reduced skeletal muscle function, e.g., the amount of force or torque exerted by a skeletal muscle group. In some embodiments, the subject exhibits reduced skeletal muscle function and orally administering a composition comprising a therapeutic nucleic acid as provided for herein retards the development of reduced skeletal muscle function, prevents deterioration of skeletal muscle function, or enhances skeletal muscle function. In some embodiment, the subject's skeletal muscle function recovers to, or is maintained at substantially the pre-treatment level after orally administering a composition comprising a therapeutic nucleic acid as provided for herein. In some embodiment, the subject exhibits an improvement in skeletal muscle function of at least about 5%, 10%, 15%, 20%, 25%, 30%35%, 40%, 50% or more, or a percentage in a range defined by any two of the preceding values, after orally administering a composition comprising a therapeutic nucleic acid as provided for herein.

In some embodiments, any of the therapeutic effects of orally administering a composition comprising a therapeutic nucleic acid as provided for herein is sustained over the duration of treatment. In some embodiments, any of the therapeutic effects of orally administering a composition comprising a therapeutic nucleic acid as provided for herein is sustained across multiple doses of administration is sustained across multiple doses of administration. In some embodiments, any of the therapeutic effects of orally administering a composition comprising a therapeutic nucleic acid as provided for herein is not transient over the duration of treatment.

In some embodiments, a treatment method of the present disclosure treats any one or more of a variety of inflammatory conditions. In some embodiments, the inflammatory condition is a chronic condition. In some embodiments, the inflammatory condition is one that is responsive to the anti-inflammatory effect of IL-10. In some embodiments, the inflammatory condition includes an autoimmune disease, graft-versus-host disease (GVHD) or an immune response to an organ transplant. In some embodiments, the inflammatory condition includes viral infection, sepsis, arthritis (rheumatoid arthritis, juvenile rheumatoid arthritis, psoriatic arthritis), multiple sclerosis, pemphigus, and type 1 diabetes (also referred to as insulin-dependent diabetes mellitus (IDDM)). In some embodiments, the inflammatory condition includes Behçet's disease, polymyositis/dermatomyositis, autoimmune cytopenias, autoimmune myocarditis, primary liver cirrhosis, Goodpasture's syndrome, autoimmune meningitis, Sjögren's syndrome, systemic lupus erythematosus, Addison's disease, alopecia greata, ankylosing spondylitis, autoimmune hepatitis, autoimmune mumps, Crohn's disease, insulin-dependent diabetes mellitus, dystrophic epidermolysis bullosa, epididymitis, glomerulonephritis, Graves' disease, Guillain-Barre syndrome, Hashimoto's disease, hemolytic anemia, multiple sclerosis, myasthenia gravis, pemphigus vulgaris, psoriasis, rheumatic fever, rheumatoid arthritis, sarcoidosis, scleroma, spondyloarthropathy, thyroiditis, vasculitis, vitiligo, myxedema, pernicious anemia and ulcerative colitis. In some embodiments, the inflammation is related to a bone marrow transplantation. In some embodiments, the inflammation is related to allograft rejection following tissue transplantation. In some embodiments, the autoimmune disease is a cardiac autoimmune disease, e.g., autoimmune myocarditis.

In some embodiments, a treatment method of the present disclosure treats symptoms and/or sequelae of any one or more of a variety of infectious diseases. In some embodiments, a heart condition or inflammatory condition treated by the nucleic acids of the present disclosure includes a symptom and/or sequelae of an infectious disease. In some embodiments, the infectious disease is associated with myocardial injury. In some embodiments, the heart condition includes acute myocarditis associated with the infectious disease. In some embodiment, the inflammatory condition includes a cytokine storm, or hyperinflammation, associate with the infectious disease. In some embodiments, the inflammatory condition includes acute lung injury or acute respiratory distress syndrome (ARDS).

In some embodiments, the infectious disease is an infection by, without limitation, one or more of the following pathogens: viruses (including but not limited to coronavirus, human immunodeficiency virus, herpes simplex virus, papilloma virus, parainfluenza virus, influenza virus, hepatitis virus, Coxsackie Virus, herpes zoster virus, measles virus, mumps virus, rubella, rabies virus, hemorrhagic viral fevers, H1N1, and the like), prions, parasites, fungi, mold, yeast and bacteria (both gram-positive and gram-negative). In some embodiments, pathogens include, without limitation, Candida albicans, Aspergillus niger, Escherichia coli (E. coli), Pseudomonas aeruginosa (P. aeruginosa), and Staphylococcus aureus (S. aureus), Group A streptococci, S. pneumoniae, Mycobacterium tuberculosis, Campylobacter jejuni, Salmonella, Shigella, and a variety of drug resistant bacteria.

In some embodiments, the inflammation is subsequent to or concurrent with an infection by a virus, e.g., a DNA or RNA virus. In some embodiments, the virus is an RNA virus, e.g., a single or double-stranded virus. In some embodiments, the RNA virus is a positive sense, single-stranded RNA virus. In some embodiments, the virus belongs to the Nidovirales order. In some embodiments, the virus belongs to the Coronaviridae family. In some embodiments, the virus belongs to the alphacoronavirus, betacoronavirus, gammacoronavirus or deltacoronavirus genus. In some embodiments, the alphacoronavirus is, without limitation, human coronavirus 229E, human coronavirus NL63 or transmissible gastroenteritis virus (TGEV). In some embodiments, the betacoronavirus is, without limitation, Severe Acute Respiratory Syndrome Coronavirus (SARS-COV), SARS-COV-2 (COVID-19), Middle Eastern Respiratory Syndrome Coronavirus (MERS-COV), human coronavirus HKU1, or human coronavirus OC43. In some embodiments, the gammacoronavirus is infectious bronchitis virus (IBV).

The nucleic acid can be administered to the subject at any suitable amount (e.g., the amount within the oral formulations as provided for herein). In some embodiments, the therapeutically effective amount of the nucleic acid includes about 0.01 μg. 0.02 μg. 0.05 μg. 0.1 μg. 0.2 μg. 0.5 μg. 1 μg. 2 μg. 3 μg. 4 μg. 5 μg. 6 μg. 7 μg. 8 μg. 9 μg. 10 μg, 15 μg, 20 μg. 25 μg. 30 μg, 40 μg, 50 μg, 75 μg, 100 μg, 125 μg, 150 μg. 175 μg, 200 μg, 250 μg, 300 μg, 400 μg. 500 μg, 600 μg. 700 μg, 800 μg, 900 μg, 1 mg, 2 mg, 3 mg, 4 mg, 5 mg, 10 mg, 15 mg, 20 mg, 30 mg, 40 mg, 50 mg, 75 mg. 100 mg or more, or an amount in a range defined by any two of the preceding values. In some embodiments, the therapeutically effective amount of the nucleic acid includes about 0.001 μg/g. 0.002 μg/g, 0.005 μg/g. 0.01 μg/g. 0.02 μg/g. 0.05 μg/g. 0.1 μg/g. 0.15 μg/g. 0.2 μg/g, 0.5 μg/g, 1 μg/g. 2 μg/g. 3 μg/g, 4 μg/g. 5 μg/g. 6 μg/g. 7 μg/g. 8 μg/g. 9 μg/g. 10 μg/g. 15 μg/g. 20 μg/g. 25 μg/g, 30 μg/g, 35 μg/g. 40 μg/g, 45 μg/g, 50 μg/g. 60 μg/g. 70 μg/g. 80 μg/g. 90 μg/g, 100 μg/g of body weight, or more, or an amount in a range defined by any two of the preceding values.

The nucleic acid or composition can be administered to the subject at any suitable dosing schedule. In some embodiments, the therapeutically effective amount of the nucleic acid or the composition is administered to the subject no more frequently than twice a week, once a week, once every two weeks, once every month, once every two months, once every three months, once every four months or longer, or at a frequency in a range defined by any two of the preceding values. In some embodiments, the nucleic acid is administered to the subject 1, 2, 3, 4, 5, 6, 7, 8, 9, 10, 15, 20, 25, 30 or more times. In some embodiments, the nucleic acid is administered to the subject at regular intervals.

While formulations are disclosed herein that can be administered using any suitable route, several embodiments of the formulations provided are unexpectedly suitable for oral delivery. Alternatively, administration can be local or systemic. In some embodiments, administration is parenteral. Suitable option for administration include, without limitation, intravenous, intramuscular, subcutaneous, intra-arterial, intraperitoneal, or oral administration. In some embodiments, the nucleic acid or composition is administered intravenously. In some embodiments, the nucleic acid or composition is administered by infusion. According to preferred embodiments, the composition is administered orally.

### Kits

Also provided herein are kits that include a nucleic acid-containing composition of the present disclosure. The present kit in some embodiments finds use in treating a muscle disorder, a heart condition or an inflammatory condition (e.g., associated with a viral infection), as provided herein. A kit can include the nucleic acid of the present disclosure and a transfection reagent. The transfections reagent can be any suitable transfection reagent, as provided herein. In some embodiments, the kit includes a pharmaceutically acceptable excipient, as provided herein. In some embodiments, the kit includes casein and/or chitosan. Kits can include one or more containers (e.g., vials, ampoules, test tubes, flasks or bottles) for holding one or more components of the kits. The kits may further include instructions for using the kit to treat a condition (e.g., HCM, HFpEF, muscular dystrophy, scleroderma, and/or an inflammatory condition associated with a viral infection). The information and instructions may be in the form of words, pictures, or both, and the like.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

### EXAMPLES

### Example 1

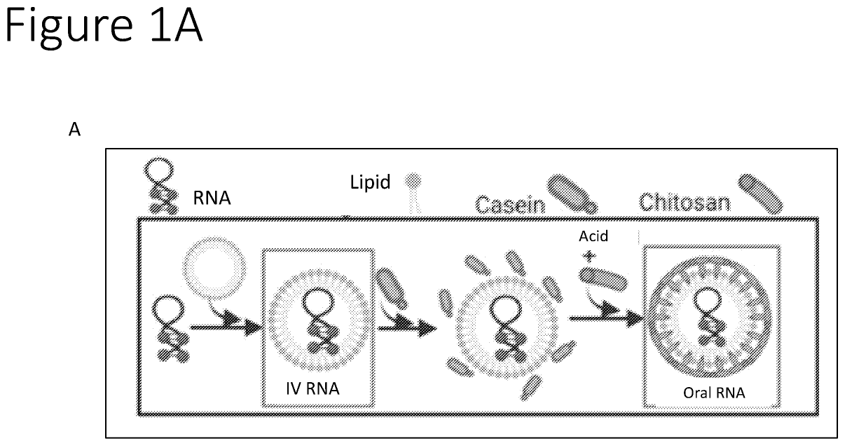

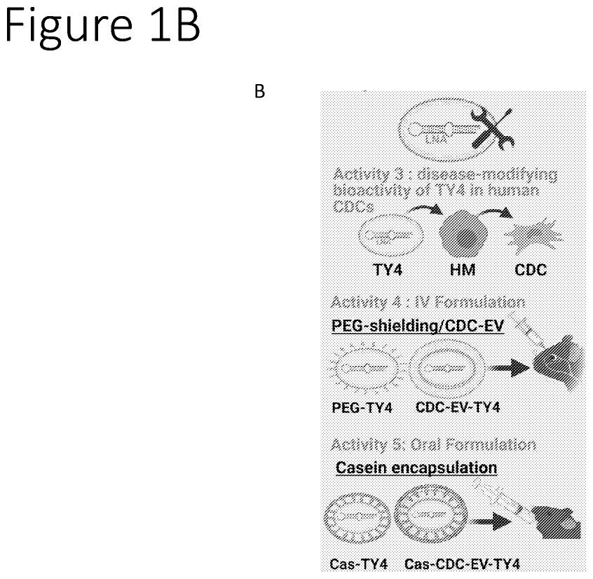

As provided for herein, several embodiments relate to the generation of compositions comprising a therapeutic nucleic acid, such as a coding or non-coding RNA, the compositions being formulated for oral administration. In several embodiments, these compositions are formulated, by way of example, according to the general schematic of FIG. 1A. FIG. 1B shows alternative embodiments. FIG. 1A depicts a non-limiting embodiment in which a nucleic acid (such as RNA, in particular a non-coding RNA with therapeutic effects upon administration) is encapsulated in an artificial lipid micelle. This encapsulated RNA is suitable for optional IV delivery. However, according to several embodiments disclosed herein, the artificial micelle is coated with casein protein. The casein-coated micelle is subsequently exposed to an acidic solution and chitosan, which results in a casein-chitosan coated micelle. The casein-chitosan coated micelle allows for oral delivery of the nucleic acid with increased bioavailability of the nucleic acid due to the casein-chitosan coated micelle imparting acid resistance to the composition, allowing it to pass through the acidic environment of the stomach with limited degradation.

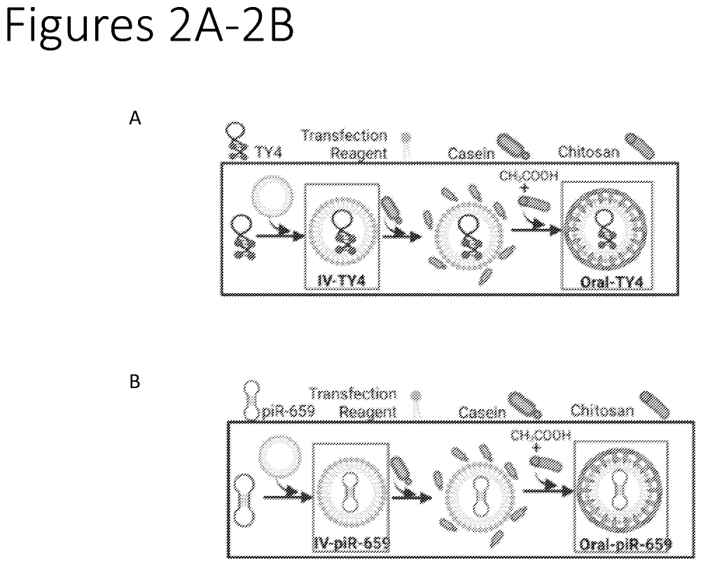

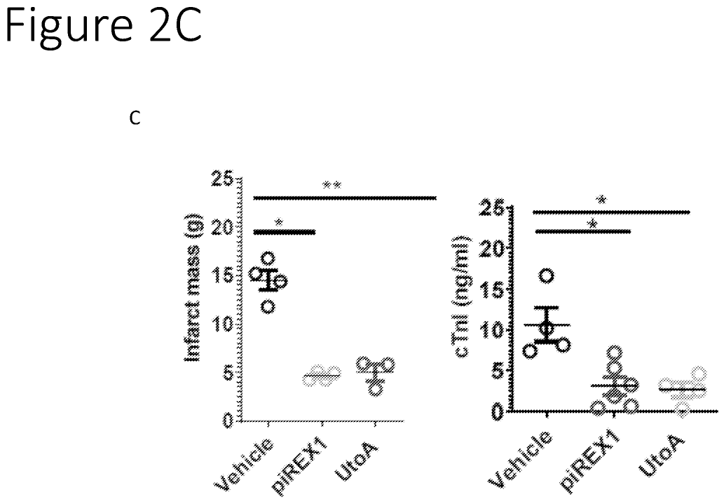

By way of non-limiting example, FIGS. 2A-2B show two examples of RNAs that were encapsulated in this fashion. FIG. 2A shows the composition comprising a TY4 RNA. FIG. 2B shows the composition of comprising a piR-659 (also referred to as piREX1). FIG. 2C shows sample data (from a myocardial infarction model) that demonstrates that encapsulated piREX1 RNA delivered orally (as well as piREX1 with a nucleic acid modification) significantly reduced infarct size as compared to animals receiving vehicle. Similarly, the circulating concentration of cardiac troponin I (a marker of cardiac injury) was significantly reduced when therapeutic piREX1 RNA was delivery orally, according to compositions provided for herein.

These data demonstrate that oral delivery of a nucleic acid, such as an RNA that yields therapeutic effects, can be accomplished using the compositions provided for herein, with enhanced therapeutic effects due to the greater bioavailability of the therapeutic RNA.

### Example 2

This non-limiting example shows the therapeutic effect of a therapeutic nucleic acid, here the non-limiting example is TY4, in particular orally administered TY4, in a model of heart failure with preserved ejection fraction (HFpEF).

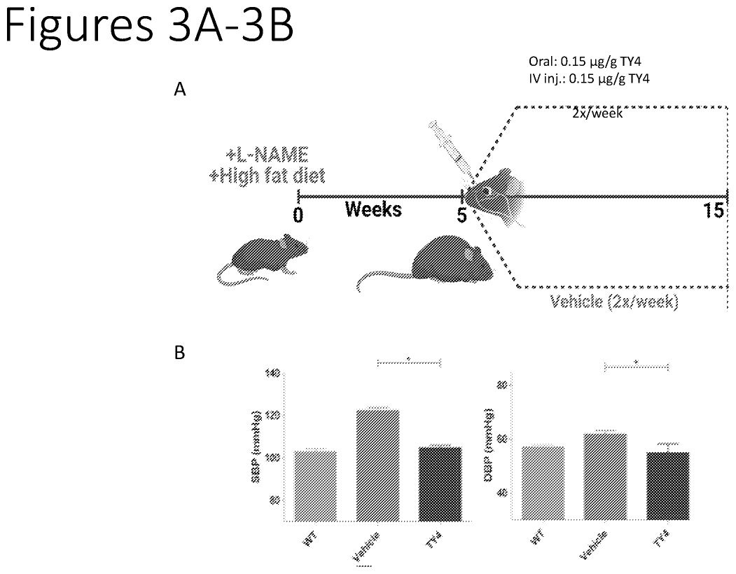

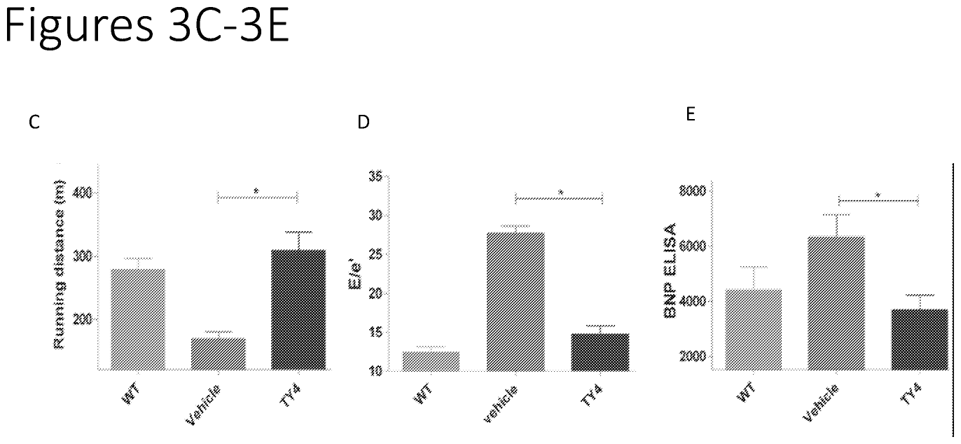

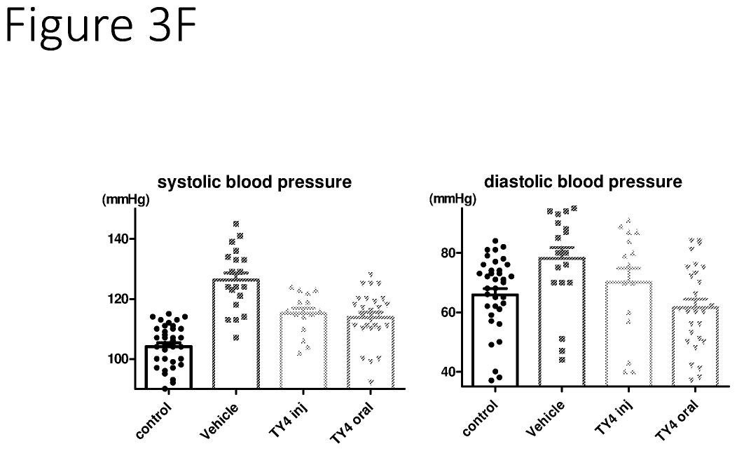

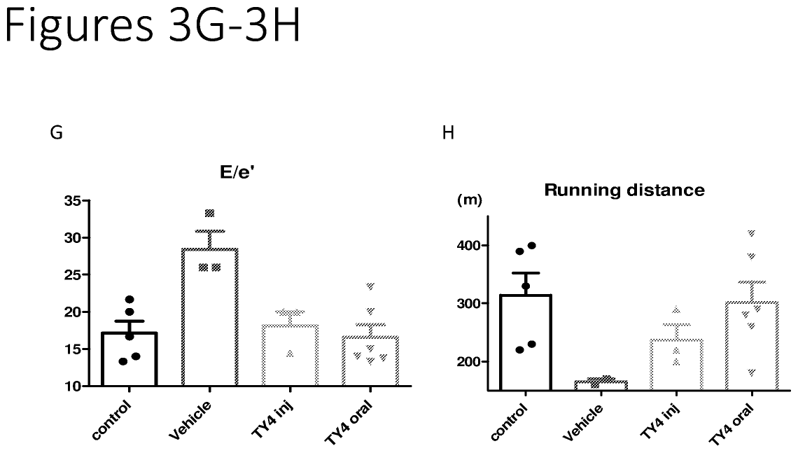

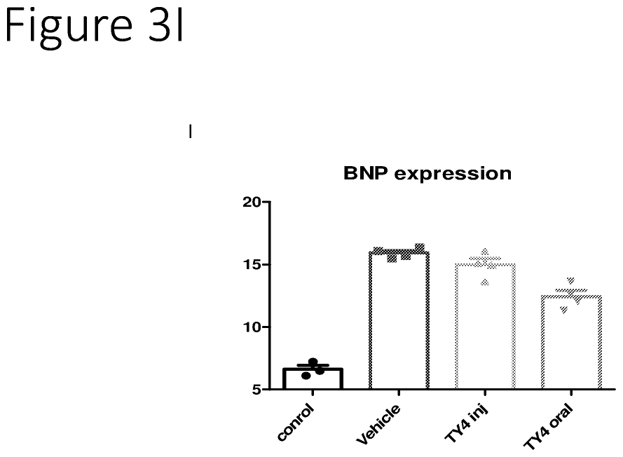

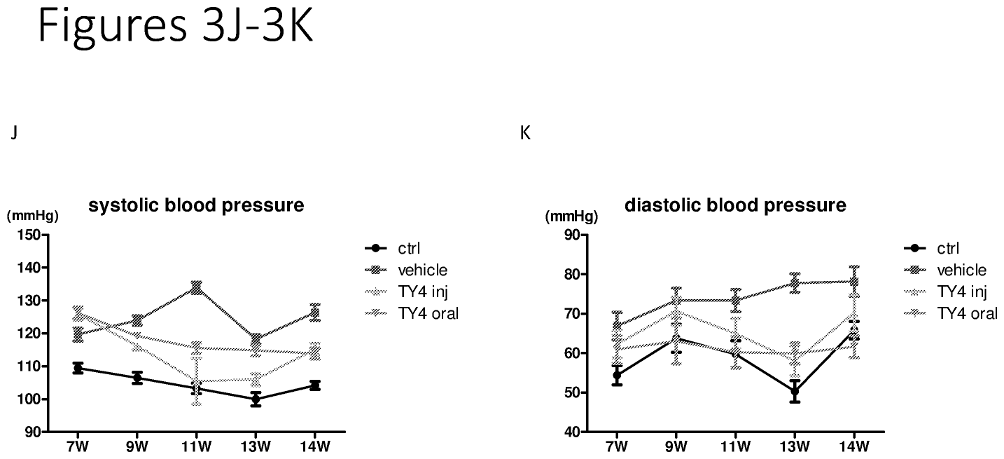

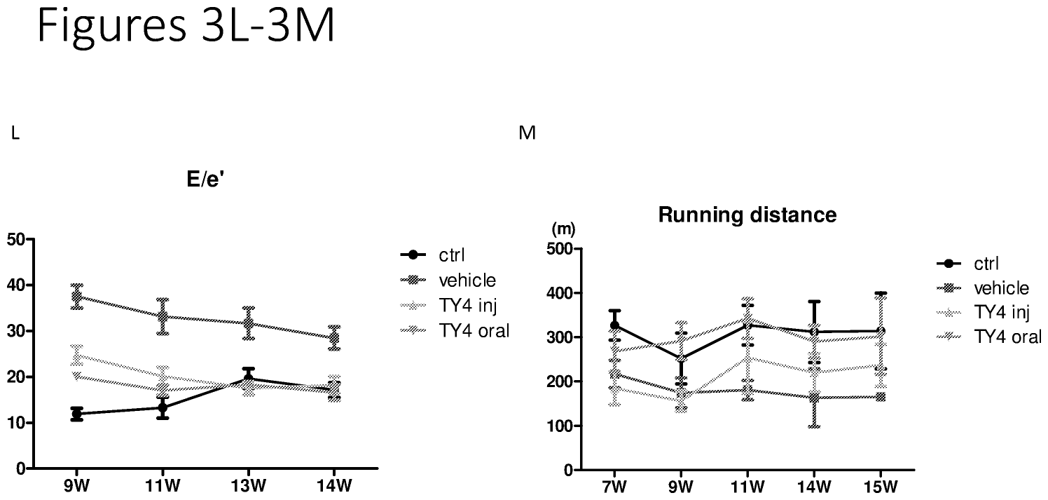

Therapeutic benefits of oral TY4 in HFpEF. Previous studies have shown the bioactivity of various RNA molecules, such as EV-YF1 in models of hypertrophy (see Example 2 of U.S. Provisional Patent Application No. 63/202,970, incorporated in its entirety by reference herein) and similar therapeutic potency of TY4 in the same model (see Example 4 of U.S. Provisional Patent Application No. 63/202,970, incorporated in its entirety by reference herein), TY4 was tested in mice with HFpEF, with oral administration investigated as well. This “two-hit” model incorporates two comorbidities commonly associated with human HFpEF (obesity and hypertension) and reproduces nitric oxide signaling abnormalities seen in heart tissue from HFpEF patients. FIG. 3A depicts the experimental protocol. Mice were either observed without intervention (WT) or fed a high-fat diet (HFD) and L-NAME-supplemented water. Within 5 weeks, the HFD/L-NAME mice become obese and hypertensive, with diastolic dysfunction but normal EF (by baseline echos). The HFpEF mice were then randomly assigned to receive twice-weekly r.o. injection (“IV Inj.”) or oral dose (“Oral”) of vehicle or TY4. TY4 was administered at 0.15 μg/g per injection or per oral administration. For oral administration, TY4 was first combined with liposomes to form a TY4-liposome complex, which was then encapsulated in a casein-chitosan complex as provided for herein. Pre-infusion/pre-oral administration and at various time points later, mice underwent blood pressure measurements, treadmill testing, echocardiography, and/or blood draws for circulating biomarkers. The only differences among groups at baseline were those associated with disease (hypertension, low exercise tolerance, elevated E/e′ ratios in HFpEF vs WT).

After 10 weeks, the following differences were evident: in TY4 animals (IV injected) blood pressure was lower (FIG. 3B), exercise tolerance was higher (FIG. 3C), E/e′ ratios were lower (FIG. 3D), and brain natriuretic peptide (BNP) levels were lower (FIG. 3E) as compared to vehicle-administered HFpEF animals. These indices of health were comparable to WT levels (non-HFpEF animals). This effect was not due to dietary aversion: the TY4 mice remained obese. The findings reveal striking disease-modifying bioactivity of TY4, especially remarkable given the refractory nature of HFpEF.

FIGS. 3A-3C: TY4 administered intravenously reverses disease progression in HFpEF mice. FIG. 3A: schematic study design. At study endpoint animals which received TY4 intravenously had lower systolic (SBP) and diastolic blood pressure (DBP) (FIG. 3B), improved exercise tolerance (FIG. 3C) and diastolic function (E/e′, FIG. 3D), and reduced levels of a serum biomarker of heart failure (BNP; FIG. 3E).

Strikingly, the oral administration of TY4 resulted in similar therapeutic effects, and in some instances superior therapeutic results. Oral administration of TY4 reduced blood pressure, both systolic and diastolic (FIG. 3F), reduced E/e′ ratios (FIG. 3G), increased exercise tolerance (FIG. 3H), and reduced BNP levels (FIG. 3I) as compared to vehicle-administered HFpEF animals. Blood pressure, exercise tolerance and E/e′ ratios were comparable to WT levels (non-HFpEF animals) in animals treated orally with TY4.

FIGS. 3F-3I: TY4 administered orally reverses disease progression in HFpEF mice. The experimental protocol is shown in FIG. 3A. At study endpoint, animals which received TY4 orally had lower systolic (SBP) and diastolic blood pressure (DBP) (FIG. 3F), improved diastolic function (E/e′, FIG. 3G) and exercise tolerance (FIG. 3H), and reduced levels of a serum biomarker of heart failure (BNP; FIG. 3I).

The therapeutic effect of TY4 was observed consistently over the course of treatment in both intravenously and orally treated animals. Reduced systolic blood pressure was observed as soon as Week 9 (FIG. 3J), while reduced diastolic pressure was observed from Week 7 onwards (FIG. 3K). Improved E/e′ ratios were observed from Week 9 and at least through Week 14 (FIG. 3L). Improved exercise tolerance was seen more consistently in orally treated animals, and all TY4-treated animals showed higher exercise tolerance by at least Week 11 (FIG. 3M).

In some embodiments, orally administering therapeutically effective amounts of a therapeutic nucleic acid, such as a non-coding RNA similar in function to TY4 to a subject having HFpEF treats the HFpEF (or one or more symptoms thereof, including without limitation, inflammation and/or fibrosis). In some embodiments, repeated oral administration of a nucleic acid, such as a non-coding RNA to a subject having HFpEF treats the HFpEF (or one or more symptoms thereof). In some embodiments, intravenously or orally administering therapeutically effective amounts of TY4 to a subject having HFpEF reduces or alleviates one or more symptoms of HFpEF. In some embodiments, intravenously or orally administering therapeutically effective amounts of TY4 to a subject having HFpEF reduces or alleviates one or more of impaired exercise endurance, elevated blood pressure, elevated E/e′ ratio, and systemic inflammation, due to the HFpEF.

### Example 3

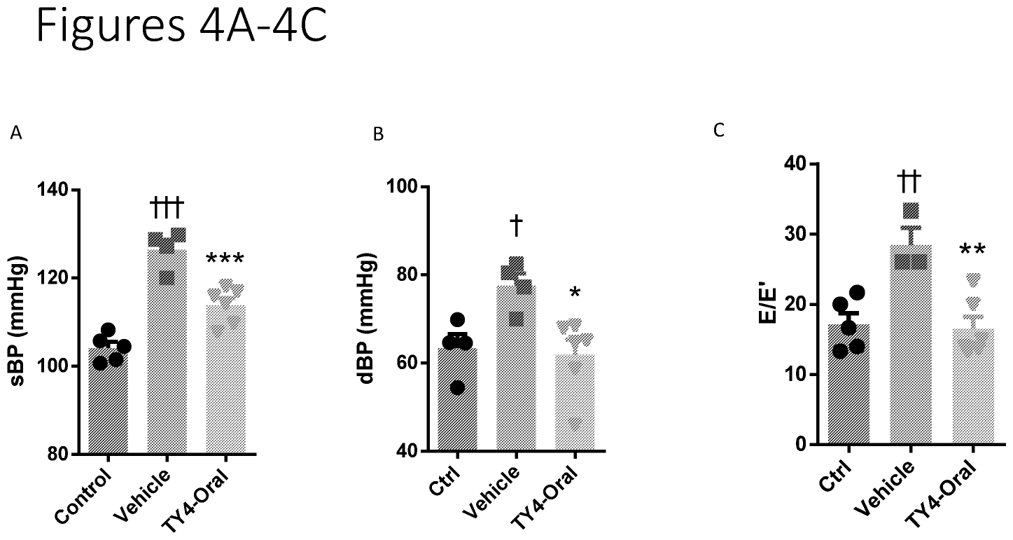

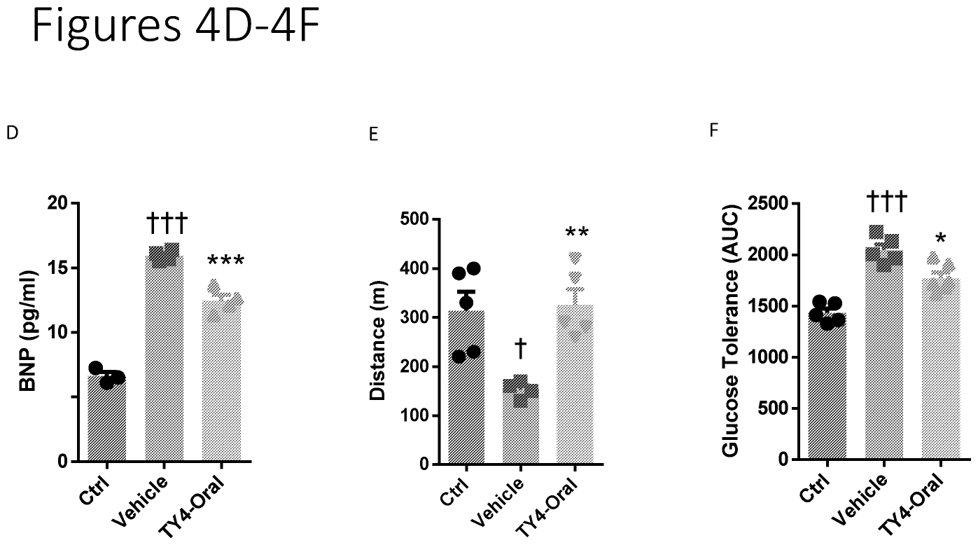

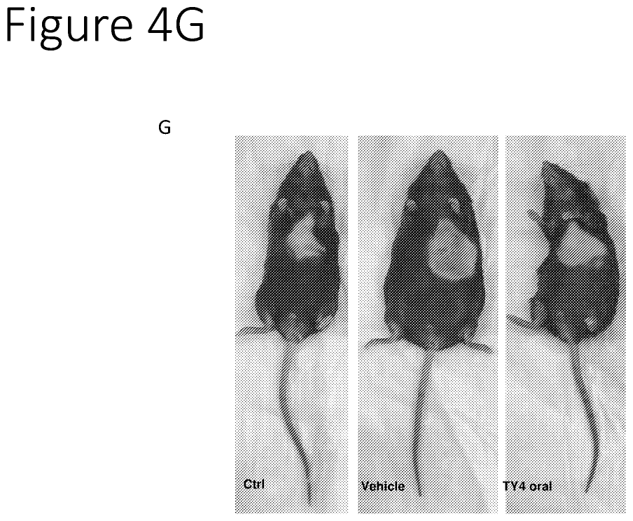

This non-limiting example represents further experimental replicates from Example 2. FIG. 4A shows data related to systolic blood pressure after delivery of a non-limiting embodiment of a therapeutic nucleic acid, TY4 RNA in this example. As shown in FIG. 4A, the oral administration of a therapeutic RNA in a casein-chitosan coated micelle results in reduced systolic blood pressure, as compared to vehicle controls (and untreated). FIG. 4B shows the coordinate reduction in diastolic blood pressure with oral administration of the therapeutic RNA. FIG. 4C shows the reduction in the E/e′ ratio after oral administration of a therapeutic RNA. FIG. 4D shows the reduction in brain natriuretic peptide as compared to vehicle controls. FIG. 4E represents the recovery in endurance, in fact to modestly greater than control levels, after oral delivery of the therapeutic RNA.

FIG. 4F shows new data beyond that of Example 2. Animals were evaluated for circulating blood glucose concentrations. As shown in FIG. 4F, treatment with vehicle alone results in elevated blood glucose concentration as compared to control. Oral administration of a nucleic acid-containing composition according to embodiments disclosed herein results in significant reductions in circulating blood glucose levels, which can be related to the obesity associated with HFpEF. FIG. 4G shows fat accumulation in the vehicle-treated mouse. As shown with the mouse on the far right, which received oral administration of the therapeutic nucleic acid, there is a reduction in fat accumulation.

Taken together, these additional and new data further reinforce that orally delivered compositions comprising therapeutic nucleic acids, such as non-coding RNA, can effectively treat HFpEF. In several embodiments, the compositions and methods provided for herein, when administered orally, can effectively reduce inflammation and/or fibrosis that are the result of, or a symptom of a disease.

### Example 4

This non-limiting example shows a study design to test different formulations for in vivo delivery of a therapeutic nucleic acid, a non-limiting example of which is TY4 and/or derivatives thereof (FIG. 1B).

PEG shielding. PEG-cationic lipid complexes (PCLC) are formed using a mixture of 2 kDa polyethylene glycol (PEG2000; 30% v/v) and Dharmafect. Complexes are formed using five freeze/thaw cycles (liquid nitrogen/60° C.) as adapted from previous preparations. A single freeze-thaw cycle involves freezing the mixture for 5 min at −190° C. (liquid nitrogen) followed by thawing for 5 at 60° C. Complexes of TY4 (and/or a derivative thereof) with PCLC are made by admixing appropriate concentrations of TY4 (and/or a derivative thereof) with 5 μl of PCLC to a final volume of 100 μl. The preparation is incubated at room temperature for 5 min with agitation.

In some embodiments, the therapeutic nucleic acid, such as TY4 (and/or a derivative thereof) is formulated as a complex with PCLC. In some embodiments, a pharmaceutical composition of a therapeutic nucleic acid, for example, TY4 (and/or a derivative thereof) includes TY4 (and/or a derivative thereof) and PCLC. In some embodiments, PEG shielding of a therapeutic nucleic acid, for example TY4 (and/or a derivative thereof), promotes oral uptake of the nucleic acid, such as an RNA, particularly a non-coding RNA, yielding therapeutic effects.

### Example 5

This non-limiting example shows formulation of CDC-EV with casein for oral administrations.

Unaltered CDC-EVs can be taken up when given orally. Casein, the dominant protein in breast milk, can enhance the uptake and bioactivity of ingested CDC-EVs, altering gene expression in blood cells and enhancing muscle function in mdx mice.

Quantifying oral uptake. Liposomes or EVs are counted as described using established NanoSight methods. A starting “dose” of 10⁷ particles is chosen. The therapeutic compound comprises a therapeutic nucleic acid (non-limiting examples include TY4 and/or a derivative thereof) is administered as is, or mixed with 8% casein solution in phosphate-buffered saline (PBS). Each therapeutic compound formulation, or the mixture of casein solution and each therapeutic compound formulation, is fed to HFpEF mice by oral gavage after 18 hours of only-food fasting, and is compared to feeding PBS alone or 8% casein solution alone after 18 hours of only-food fasting. One hour after oral administration of each test article, blood is collected from the inferior vena cava for RNA extraction. Uptake into the blood is quantified by measuring therapeutic compound levels in whole blood by qPCR, using RNA isolation methods. Measured PCR cycles are compared against standards created by spiking known levels of TY4 into mouse blood. TY4 is derived from a human-specific sequence, so background levels in mice are below the limit of reliable PCR detection. Selection for further characterization is based upon measured levels of TY4 in blood. A formulation (whether with or without casein) can be considered to provide oral delivery if TY4 is detected by 2 amplification cycles [Ct] of control or earlier by qPCR. In some embodiments, a formulation provides detection at >3 Ct lower than the nearest competitor by qPCR. A formulation may be further tested for in vivo bioactivity.

In vivo bioactivity. To assess disease-modifying bioactivity, the mouse HFpEF model is used. Starting 5 weeks after the initiation of high-fat diet+L-NAME, HFpEF mice will be fed vehicle (PBS), and each of the formulations advanced (with or without casein), every other day.

In some embodiments, therapeutic nucleic acids, including non-coding RNA, such as TY4 (and/or a derivative thereof) (in liposomes or CDC-EV) is formulated with casein. In some embodiments, a pharmaceutical composition of TY4 (and/or a derivative thereof) includes TY4 (and/or a derivative thereof) in liposomes or CDC-EV, and casein. In some embodiments, a pharmaceutical composition of TY4 (and/or a derivative thereof) includes TY4 (and/or a derivative thereof) in liposomes or CDC-EV, and 8% casein. In some embodiments, a formulation of TY4 (and/or a derivative thereof) (in liposomes or CDC-EV) and casein, e.g., 8% casein, promotes oral uptake of TY4 (and/or a derivative thereof).

### Example 6

This non-limiting example shows oral formulations comprising therapeutic nucleic acids encapsulated in lipid micelles and coated with a casein-chitosan complex ameliorate symptoms of myocardial infarction.

To assess the effectiveness of oral delivery of a therapeutic nucleic acid, myocardial infarction was modeled in mice by open-chest occlusion of the left anterior descending coronary artery for 45 min, followed by reperfusion. The chest was then closed. Twenty min after reperfusion, mice were given either vehicle or an IV composition made up of a lipid-encapsulated therapeutic RNA, or oral composition comprising a therapeutic RNA encapsulated in a lipid micelle and coated with casein-chitosan, as provided for herein. The non-limiting example of a nucleic acid payload use here was TY4. Hearts were excised 48 hours post-MI and infarct size (IS) quantified histologically.

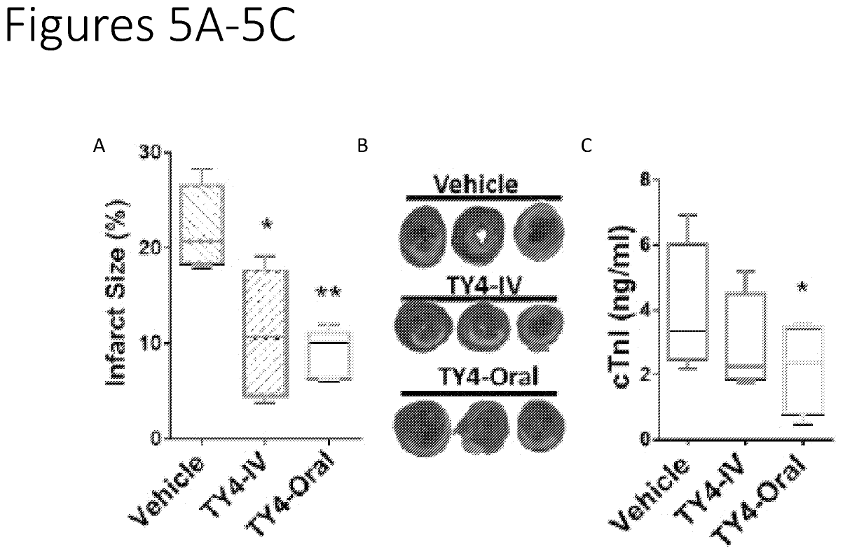

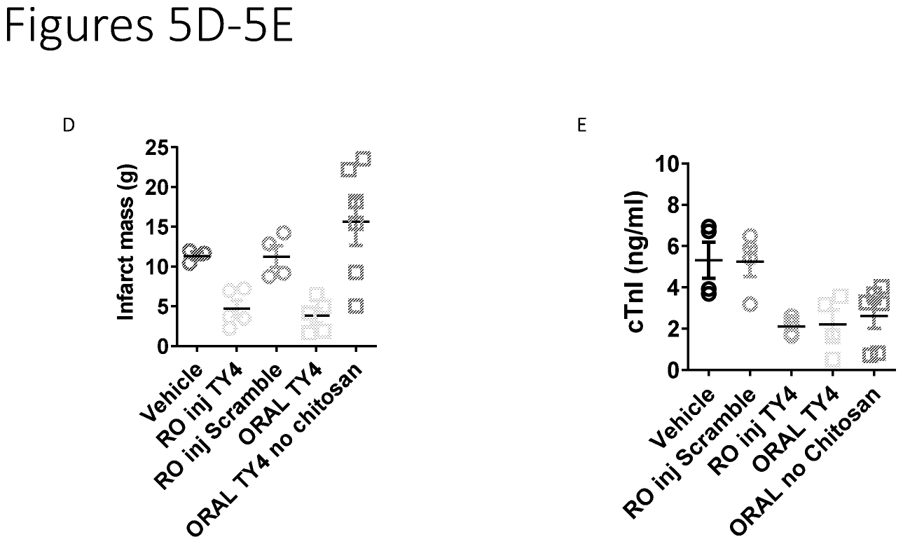

FIG. 5A shows pooled data related to infarct size. As shown, both IV and oral compositions resulted in reduced infarct size. Notably, the oral delivery of TY4 shows an enhanced reduction of infarct size. Representative left ventricular sections for each group are shown in FIG. 5B, with the orally-treated group showing markedly less infarct scarring. As a marker of cardiac injury, FIG. 5C shows that orally administered TY4 yielded a significant decrease as compared to control. Taken together, the data for histology and troponin I are mutually-reinforcing in showing the cardioprotective efficacy of orally delivered therapeutic nucleic acids, such as the non-coding RNA TY4.

Further building on those finds, additional comparative analysis is shown in FIGS. 5D and 5E. FIG. 5D shows IV injection of TY4 (or a scrambled version thereof) or orally administered TY4 housed in compositions according to embodiments disclosed herein (e.g., micelles coated with casein-chitosan). An additional group here includes oral TY4 encapsulated in a micelle that is coated with casein alone (no chitosan). The data of FIG. 5D reinforce the findings discussed above with respect to oral delivery of a therapeutic RNA, but also demonstrate that, according to preferred embodiments, a lipid micelle is coated with both casein and chitosan. The test group on the right of FIG. 5D is the RNA-encapsulated micelles coated with casein only. Infarct size for that group was notably increased, indicative of a reduced bioavailability of the TY4, believed to be due to less robust protection for the composition in the low-acid environment of the stomach. FIG. 5E shows less of a drop off in efficacy, as related to measuring cardiac troponin I, though the casein-only formulation appears to at least trend towards elevated concentrations (less therapeutic effect). Taken together, these data support the cardioprotective efficacy of orally delivered therapeutic nucleic acids, such as the non-coding RNA TY4, using a casein-chitosan coating, as provided for herein in several embodiments.

### Example 7

This non-limiting example shows oral formulations comprising therapeutic nucleic acids encapsulated in lipid micelles and coated with a casein-chitosan complex improve symptoms of scleroderma.

Scleroderma is an autoimmune disorder marked by progressive skin thickening and fibrosis of skin, heart and lung. An animal model of scleroderma was used in which mice were injected with bleomycin intradermally over the course of 3 weeks. Animals were then treated with compositions configured for oral delivery of therapeutic RNA molecules, as provided for herein. This experiment uses a non-limiting example RNA, TY4, which is encapsulated in a lipid micelle coated with casein-chitosan and delivered orally. Oral delivery (in the same delivery composition) of a scrambled RNA sequence was used as a control.

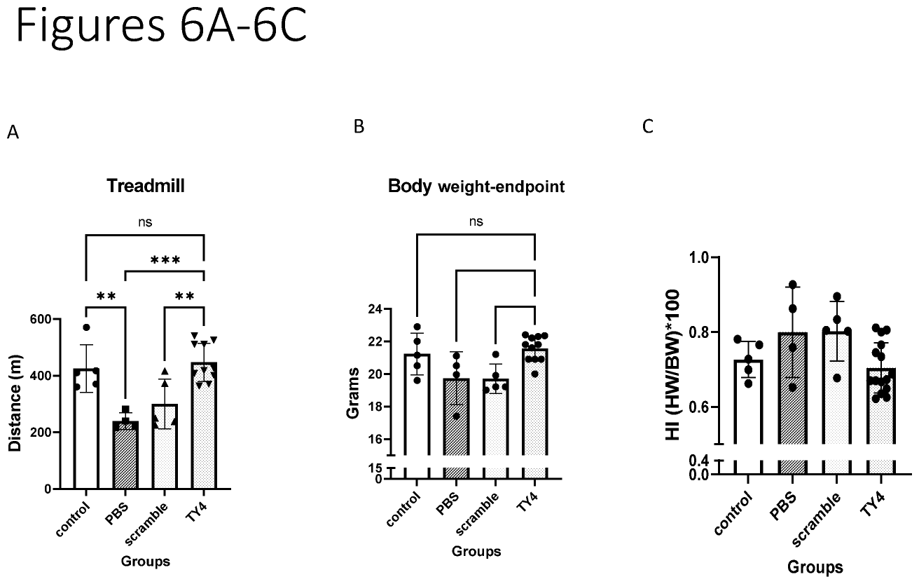

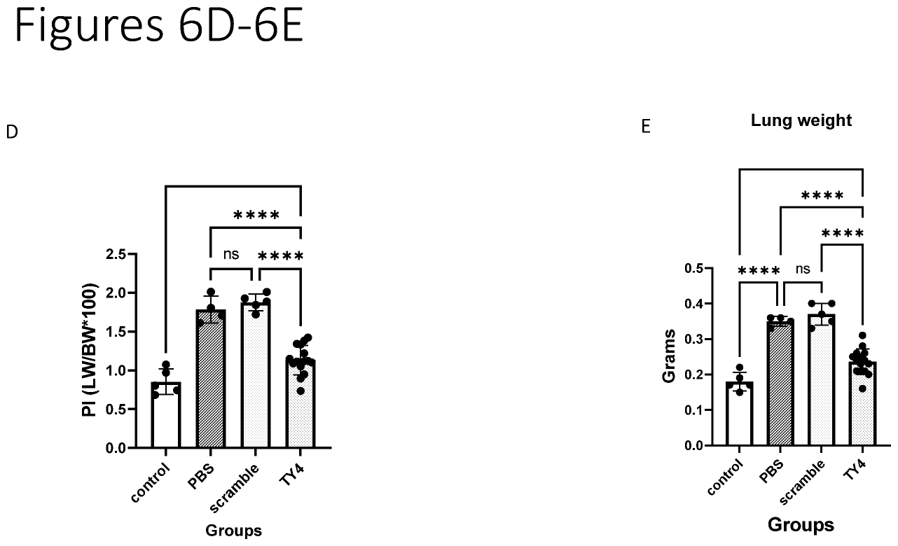

FIG. 6A shows data related to endurance testing on a treadmill. As shown, oral delivery of the therapeutic RNA resulted in full return of endurance to that of untreated control mice, PBS control and scrambled RNA sequence controls each showed significant reductions in endurance. FIG. 6B shows that oral delivery of the therapeutic RNA allowed mice to maintain body weight such that it was not significantly different from control. FIG. 6C shows a heart index (HI) that relates the heart weight to the body weight of the mice in each group. As expected from the reduction in body weight with the non-therapeutic groups, these groups exhibited an elevated HI. Also, an increase in heart weight (e.g., due to fibrosis) could also account for an aspect of the increased HI. Likewise, when measuring pulmonary index (PI), which indexes lung weight as a function of body weight, the orally delivered therapeutic RNA results in significantly reduced PI as compared to the non-therapeutic groups (though the PI was still elevated over control). FIG. 6E shows the lung weight data alone, which corresponds to the PI data.

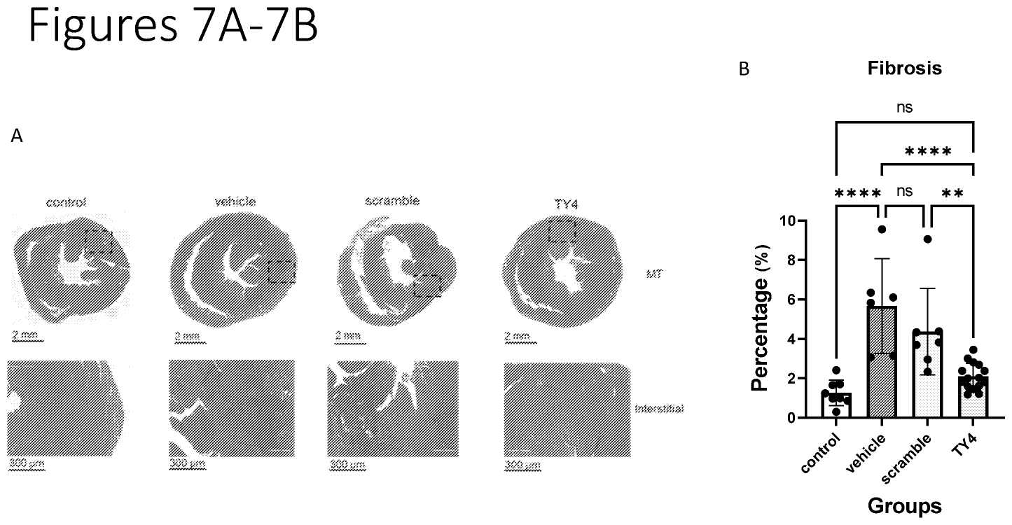

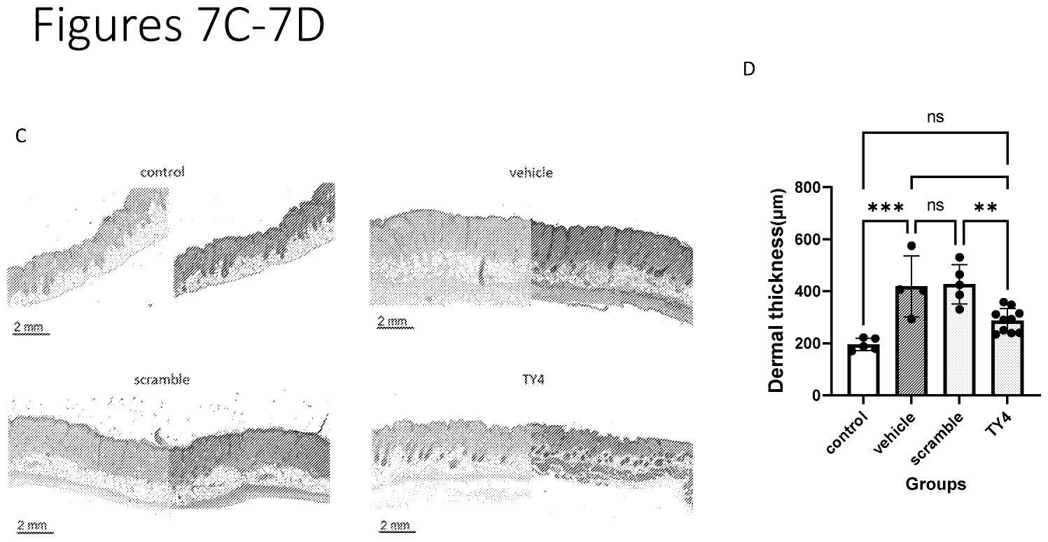

Turning specifically to cardiac measures, FIG. 7A shows histology data related to fibrosis. The top row shows representative tissue stains with dashed boxes corresponding to the enlarged view provided in the second row. The orally delivered therapeutic RNA shows a far reduced fibrosis of the tissue. FIG. 7B shows the quantification of fibrosis for each group, indicating that oral delivery of the therapeutic RNA results in significantly reduced cardiac fibrosis. Turning to symptoms of scleroderma that impact the skin, FIGS. 7C and 7D relate to fibrosis of the skin. FIG. 7C show histology data related to the skin with the upper left showing control skin, upper right showing vehicle control, lower left showing the scrambled RNA, and lower right showing the orally delivered TY4 RNA. The orally delivered TY4 RNA, as a non-limiting example of a therapeutic RNA. resulted in visibly less thickening/fibrosis of the skin as compared to the non-treatment groups. FIG. 7D confirms this with a graph of derma thickness for each group, with orally delivered therapeutic RNA resulting in dermal thickness that is not significantly different from control.

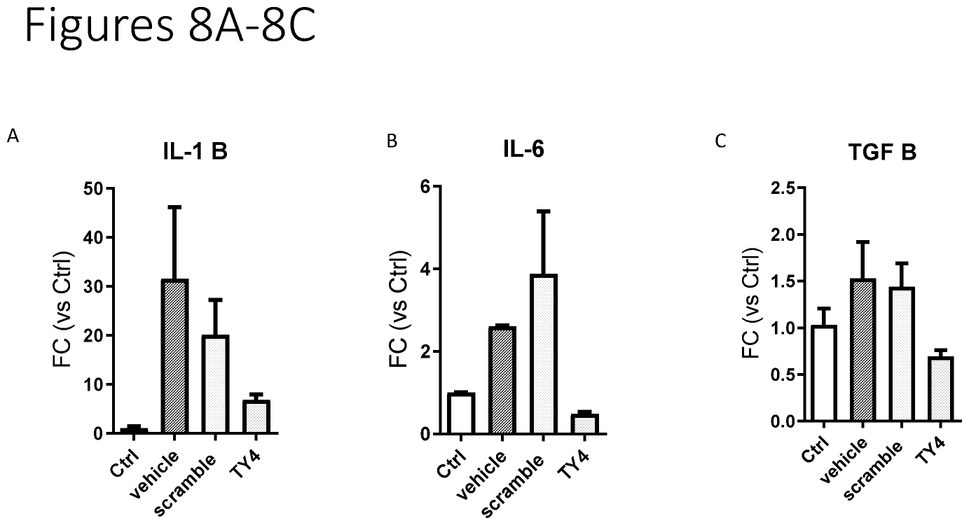

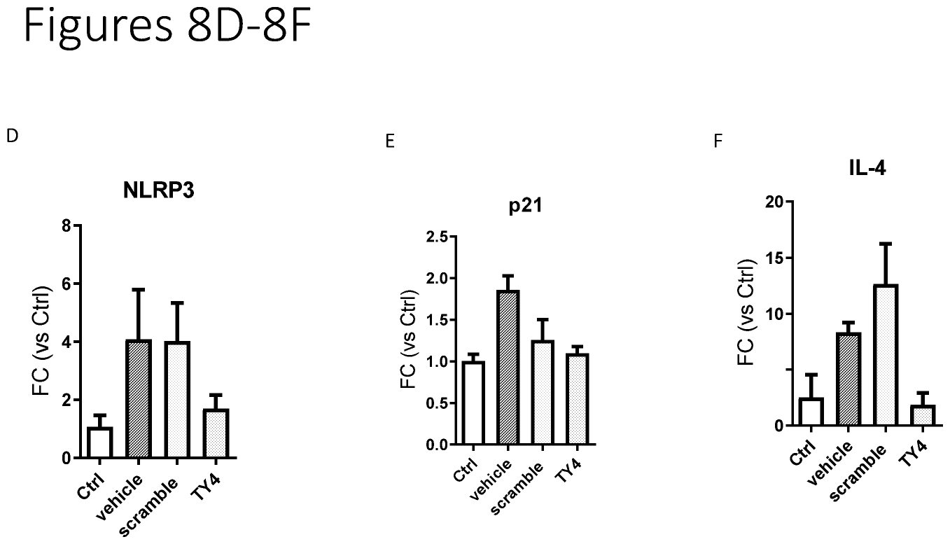

Additional investigation was undertaken with respect to the expression levels of various inflammatory cytokines. FIGS. 8A-8F show qPCR data related to quantification of IL1-B (8A), IL-6 (8B), TGF beta (8C), NLRP3 (8D), p21 (8E), and IL-4 (8F). Each of these cytokines was equivalent to or showed only modest increases in expression when the orally delivered therapeutic RNA was administered. These data thus support the efficacy of oral delivery of a therapeutic RNA to reduce fibrosis and/or inflammatory conditions, such as those secondary or symptomatic of scleroderma.

### Example 8

This non-limiting example shows oral formulations comprising therapeutic nucleic acids encapsulated in lipid micelles and coated with a casein-chitosan complex improve symptoms of muscular dystrophy.

To determine the effects of orally delivered therapeutic RNA, twelve-fourteen-month-old female mdx mice were fed TY4 (0.15 μg/g body weight) or vehicle twice-weekly for 8 weeks. Cardiac and skeletal muscle function were measured prior to feeding (i.e., baseline) and at the 8-week study endpoint. Hearts and tibialis anterior (TA) muscles were dissected and processed for Masson's trichrome staining to quantify interstitial fibrosis.

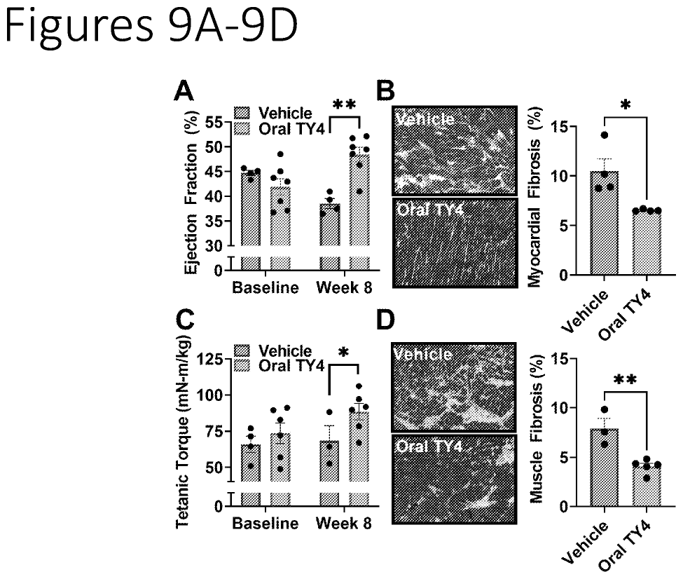

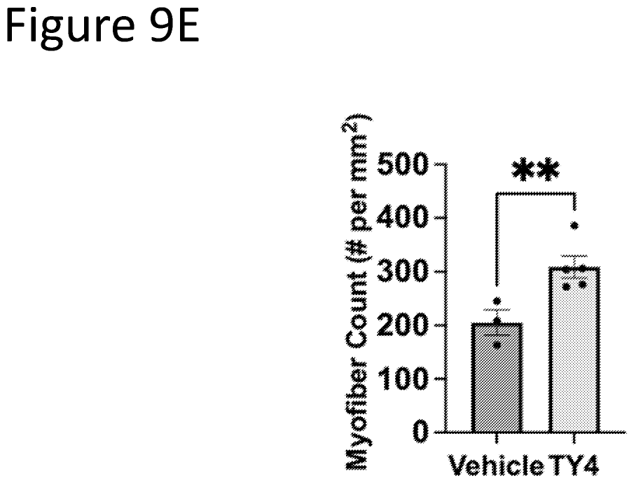

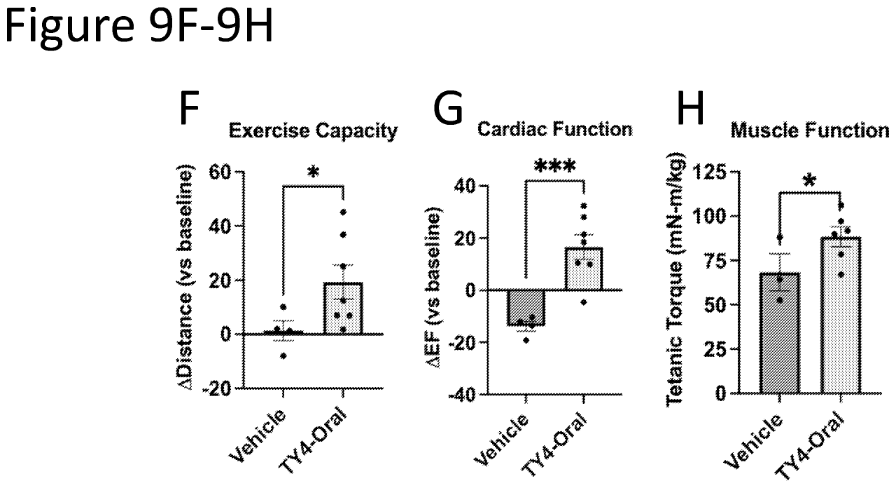

As shown in FIG. 9A, transthoracic echocardiography on lightly anesthetized mdx mice was performed to measure left ventricular ejection fraction (EF). At baseline, no differences were detected between groups. After 8 weeks, mdx receiving the orally administered example of a therapeutic RNA, TY4, had higher EF relative to vehicle control, which declined during the study period. As shown in FIG. 9B, Masson's trichrome micrographs and pooled data (right subpanel) show mdx mice receiving orally administered TY4 had less myocardial fibrosis than vehicle control mice. Turning to in vivo muscle function of the anterior crural muscles, of which the tibialis anterior (TA) produces ˜85% of the torque output for this muscle group, was recorded by attaching the foot of the mdx mouse to an aluminum shoe (which was attached to the servomotor of a force transducer) and stimulating the left common peroneal nerve. Tetanic torque was recorded at 200 Hz. At baseline, no differences were detected between groups (see FIG. 9C). After 8 weeks, mdx mice receiving orally administered TY4 produced more torque than vehicle control mice.

FIG. 9D relates to muscle fibrosis and shows Masson's trichrome micrographs and pooled data (right subpanel). These data indicate that mdx mice receiving orally administered TY4 had less muscle fibrosis than vehicle control mice. FIG. 9E summarizes physiological data collected that demonstrates that there is a greater myofiber count (per mm²).

Functional data that were collected further support the efficacy of orally administered therapeutic RNA by way of delivery using a lipid micelle encapsulating the therapeutic RNA and coated with a casein-chitosan complex. FIG. 9E shows that in addition to the decreased interstitial fibrosis (FIG. 9C/9D) orally administered therapeutic RNA by way of delivery using a lipid micelle encapsulating the therapeutic RNA boosted the number of myofibers in the TA. This physiologic effect was confirmed in functional assays (FIGS. 9F-9H) which show enhance exercise capacity, cardiac function and muscle function, respectively, in animals that received oral administration of a therapeutic RNA by way of delivery using a lipid micelle encapsuling the therapeutic RNA. Two-way ANOVA or an independent t-test was used to determine statistical significance between groups. *P<0.05. ** P<0.01. Data are represented as mean±SEM.

These data further support the efficacy of orally administered therapeutic RNA by way of delivery using a lipid micelle encapsuling the therapeutic RNA and coated with a casein-chitosan complex. In several embodiments, such compositions to deliver therapeutic RNA via an oral administration result in increased bioavailability of the RNA, which in turn leads to enhanced therapeutic outcomes for diseases hallmarked by inflammation and/or fibrosis, such as muscular dystrophy.

Although the foregoing has been described in some detail by way of illustrations and examples for purposes of clarity and understanding, it will be understood by those of skill in the art that modifications can be made without departing from the spirit of the present disclosure. Therefore, it should be understood that the forms disclosed herein are illustrative only and are not intended to limit the scope of the present disclosure, but rather to also cover all modification and alternatives coming with the true scope and spirit of the embodiments of the present disclosure.

It is contemplated that various combinations or subcombinations of the specific features and aspects of the embodiments disclosed above may be made. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed subject matter. Thus, it is intended that the scope of the present disclosure should not be limited by the particular disclosed embodiments described above. Moreover, while the disclosed subject matter is susceptible to various modifications, and alternative forms, specific examples thereof have been shown in the drawings and are herein described in detail. It should be understood, however, that the present disclosure is not to be limited to the particular forms or methods disclosed, but is to cover all modifications, equivalents, and alternatives falling within the spirit and scope of the various embodiments described and the appended claims.

Any methods disclosed herein need not be performed in the order recited. The methods disclosed herein include certain actions taken by a practitioner; however, they can also include any third-party instruction of those actions, either expressly or by implication. For example, actions such as “administering to a subject in need of treating a heart condition or symptom thereof a therapeutically effective amount of the nucleic acid” include “instructing the administration of an effective amount of the nucleic acid to a subject.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than.” “less than,” “between,” and the like includes the number recited. Numbers preceded by a term such as “about” or “approximately” include the recited numbers. For example, “about 90%” includes “90%.” In some embodiments, at least 95% identical includes 96%, 97%, 98%. 99%, and 100% identical to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like.

All patents and other publications; including literature references, issued patents, published patent applications, and co-pending patent applications cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicant and does not constitute any admission as to the correctness of the dates or contents of these documents.

What is claimed is:

1. A formulation for oral delivery of a nucleic acid, comprising:
a nucleic acid;
a cationic lipid;
at least one casein protein; and
a chitosan.

2. The formulation of claim 1, wherein
the nucleic acid comprises a ribonucleic acid (RNA) and wherein the RNA is present in an amount ranging between about 0.0001 and 0.01% of the formulation by weight per volume;
wherein the at least one casein protein comprises at least an α-s1 casein subunit and wherein the at least one casein protein is present in an amount ranging between about 0.5 and 5% of the formulation by weight per volume; and
wherein the chitosan is present in an amount ranging between about 0.001 and 1% of the formulation by weight per volume.

3. The formulation of claim 2, further comprising acetic acid, wherein the acetic acid is present in an amount ranging between about 0.01 and 1% of the formulation by weight per volume.

4. The formulation of claim 2 wherein the cationic lipid is present in an amount ranging from about 0.1 to about 5 microliters for each microgram of nucleic acid.

5. The formulation of claim 2, wherein the nucleic acid comprises a non-coding RNA.

6. The formulation of claim 2, further comprising an acid.

7. The formulation of claim 6, wherein the acid is present in an amount ranging between about 0.001 and 1% of the formulation by volume and where the acid is selected from acetic acid, citric acid, phosphoric acid and citric acid.

8. The formulation of claim 2, wherein the chitosan is low molecular weight chitosan.

9. The formulation of claim 8, wherein the low molecular weight chitosan ranges in mass from about 50 to about 190 kiloDaltons.

10. The formulation of claim 2, wherein the nucleic acid comprises a non-coding RNA, wherein the non-coding RNA is present in an amount ranging from between about 0.001 and about 0.005% of the formulation by weight per volume,
wherein the at least one casein protein comprises a mixture of an α-s1 casein subunit, an α-s2 casein subunit, a β casein subunit, and a κ casein subunit, wherein the casein subunits are present in an amount ranging between about 1 and 3% of the formulation by weight per volume; and
wherein the chitosan is present in an amount ranging between about 0.01 and 0.1% of the formulation by weight per volume.

11. The formulation of claim 10, wherein the non-coding RNA is present in an amount ranging from between about 0.0015 and about 0.004% of the formulation by volume,
wherein the mixture of casein subunits are present in an amount ranging between about 2 and 3% of the formulation by weight per volume;
wherein the chitosan is present in an amount ranging between about 0.05 and 0.1% of the formulation by weight per volume; and
wherein the cationic lipid is present in an amount ranging from about 1 to about 3 microliters for each microgram of nucleic acid.

12. The formulation of claim 11, wherein the non-coding RNA is present in an amount ranging from between about 0.0015 and about 0.0035% of the formulation by volume,
wherein the mixture of casein subunits are present in an amount ranging between about 2.2 and 2.8% of the formulation by weight per volume;
wherein the chitosan is present in an amount ranging between about 0.06 and 0.09% of the formulation by weight per volume; and
wherein the cationic lipid is present in an amount ranging from about 1 to about 2 microliters for each microgram of nucleic acid.

13. The formulation of claim 12, wherein, upon administration to a subject, the non-coding RNA reduces expression of one or more of IL1-B, IL-6, TGF beta, NLRP3, p21, and IL-4.

14. The formulation of claim 12, wherein, upon administration to a subject, the non-coding RNA reduces systolic blood pressure of the subject.

15. The formulation of claim 2, wherein, upon administration to a subject, the non-coding RNA reduces diastolic blood pressure of the subject.

16. The formulation of claim 12, wherein, upon administration to a subject, the non-coding RNA enhances muscular endurance, muscular resistance to fatigue, muscular strength and/or muscle contractility of at least one muscle of the subject.

17. The formulation of claim 16, wherein, the muscle is skeletal muscle or cardiac muscle.

18. The formulation of claim 12, wherein, upon administration to a subject, the non-coding RNA reduces the expression of brain natriuretic peptide.

19. The formulation of claim 12, wherein, upon administration to a subject, the non-coding RNA reduces diastolic mitral inflow velocity to mitral annular tissue velocity (E/e′).

20. The formulation of claim 12, wherein, upon administration to a subject, the non-coding RNA enhances glucose tolerance within the subject.

21. The formulation of claim 12, wherein, upon administration to a subject, the non-coding RNA reduces obesity and/or subcutaneous adipose tissue per unit body mass of the subject.

22. The formulation of claim 12, wherein, upon administration to a subject having had a myocardial infarction, the non-coding RNA reduces infarct size after the myocardial infarction.

23. The formulation of claim 12, wherein, upon administration to a subject having had a myocardial infarction, the non-coding RNA reduces circulating cardiac troponin I concentration after the myocardial infarction.

24. A formulation according to any one of claims 1 to 23, wherein the formulation alleviates one or more symptoms of a disease associated with increased inflammation and/or fibrosis.

25. The formulation of claim 24, wherein the disease is selected from heart failure with preserved ejection fraction, myocardial infarction, muscular dystrophy, scleroderma, viral infection, and hypertrophic cardiomyopathy.

26. A formulation according to any one of claims 1 to 25, wherein the nucleic acid comprises a sequence having at least 90% sequence identity to one or more of SEQ ID NO: 1-25, 31, 32.

27. A formulation according to any one of claims 1 to 26, wherein the nucleic acid consists essentially of a sequence having at least 90% sequence identity to one or more of SEQ ID NO: 1-25, 31, 32.

28. A formulation for oral delivery of a nucleic acid, comprising:
an artificial lipid micelle,
a nucleic acid, wherein the nucleic acid is encapsulated within the artificial lipid micelle, and
a coating on the artificial lipid micelle, wherein the coating comprises a mixture of casein proteins and chitosan polymers.

29. The formulation of claim 28, wherein
the nucleic acid comprises a ribonucleic acid (RNA) and wherein the RNA is present in an amount ranging between about 0.0001 and 0.01% of the formulation by weight per volume;
wherein the mixture of casein proteins and chitosan polymers comprises at least an α-s1 casein subunit and wherein the at least one casein protein is present in an amount ranging between about 0.5 and 5% of the formulation by weight per volume; and
wherein the chitosan is present in an amount ranging between about 0.001 and 1% of the formulation by weight per volume.

30. The formulation of claim 28 or 29, further comprising acetic acid, wherein the acetic acid is present in an amount ranging between about 0.01 and 1% of the formulation by weight per volume.

31. The formulation of claim 28, 29, or 30, wherein the cationic lipid is present in an amount ranging from about 0.1 to about 5 microliters for each microgram of nucleic acid.

32. A formulation according to any one of claims 28 to 31, wherein the nucleic acid comprises a non-coding RNA.

33. The formulation of claims 28, 29 or 31 to 32, further comprising an acid.

34. The formulation of claim 33, wherein the acid is present in an amount ranging between about 0.001 and 1% of the formulation by volume and where the acid is selected from acetic acid, citric acid, phosphoric acid and citric acid.

35. A formulation according to any one of claims 28 to 34, wherein the chitosan is low molecular weight chitosan.

36. The formulation of claim 35, wherein the low molecular weight chitosan ranges in mass from about 50 to about 190 kiloDaltons.

37. The formulation of claim 29, wherein the nucleic acid comprises a non-coding RNA, wherein the non-coding RNA is present in an amount ranging from between about 0.001 and about 0.005% of the formulation by volume,
wherein the at least one casein protein comprises a mixture of an α-s1 casein subunit, an α-s2 casein subunit, a β casein subunit, and a κ casein subunit, wherein the casein subunits are present in an amount ranging between about 1 and 3% of the formulation by volume; and
wherein the chitosan is present in an amount ranging from about 1 to about 3 microliters for each microgram of nucleic acid e.

38. The formulation of claim 37, wherein the non-coding RNA is present in an amount ranging from between about 0.0015 and about 0.005% of the formulation by volume,
wherein the mixture of casein subunits are present in an amount ranging between about 2 and 3% of the formulation by volume;
wherein the chitosan is present in an amount ranging between about 0.05 and 0.1% of the formulation by volume; and
wherein the cationic lipid is present in an amount ranging from about 1 to about 2 microliters for each microgram of nucleic acid.

39. The formulation of claim 37 or 38, wherein the non-coding RNA is present in an amount ranging from between about 0.0015 and about 0.0035% of the formulation by volume,
wherein the mixture of casein subunits are present in an amount ranging between about 2.2 and 2.8% of the formulation by volume;
wherein the chitosan is present in an amount ranging between about 0.06 and 0.09% of the formulation by volume; and
wherein the cationic lipid is present in an amount ranging from about 15 to about 25 ng per microliter of the formulation.

40. A formulation according to any one of claims 28 to 39, wherein, upon administration to a subject, the non-coding RNA reduces expression of one or more of IL1-B, IL-6, TGF beta, NLRP3, p21, and IL-4.

41. A formulation according to any one of claims 28 to 40, wherein, upon administration to a subject, the non-coding RNA reduces systolic blood pressure of the subject.

42. A formulation according to any one of claims 28 to 41, wherein, upon administration to a subject, the non-coding RNA reduces diastolic blood pressure of the subject.

43. A formulation according to any one of claims 28 to 42, wherein, upon administration to a subject, the non-coding RNA enhances muscular endurance, muscular resistance to fatigue, muscular strength and/or muscle contractility of at least one muscle of the subject.

44. The formulation of claim 43, wherein, the muscle is skeletal muscle or cardiac muscle.

45. A formulation according to any one of claims 28 to 44, wherein, upon administration to a subject, the non-coding RNA reduces the expression of brain natriuretic peptide.

46. A formulation according to any one of claims 28 to 45, wherein, upon administration to a subject, the non-coding RNA reduces diastolic mitral inflow velocity to mitral annular tissue velocity (E/e′).

47. A formulation according to any one of claims 28 to 46, wherein, upon administration to a subject, the non-coding RNA enhances glucose tolerance within the subject.

48. A formulation according to any one of claims 28 to 47, wherein, upon administration to a subject, the non-coding RNA reduces obesity and/or subcutaneous adipose tissue per unit body mass of the subject.

49. A formulation according to any one of claims 28 to 48, wherein, upon administration to a subject having had a myocardial infarction, the non-coding RNA reduces infarct size after the myocardial infarction.

50. A formulation according to any one of claims 28 to 48, wherein, upon administration to a subject having had a myocardial infarction, the non-coding RNA reduces circulating cardiac troponin I concentration after the myocardial infarction.

51. A formulation according to any one of claims 28 to 50, wherein the formulation alleviates one or more symptoms of a disease associated with increased inflammation and/or fibrosis.

52. The formulation of claim 51, wherein the disease is selected from heart failure with preserved ejection fraction, myocardial infarction, muscular dystrophy, scleroderma, viral infection, and hypertrophic cardiomyopathy.

53. A formulation according to any one of claims 28 to 52, wherein the nucleic acid comprises a sequence having at least 90% sequence identity to one or more of SEQ ID NO: 1-25, 31, 32.

54. A formulation according to any one of claims 1 to 25, wherein the nucleic acid consists essentially of a sequence having at least 90% sequence identity to one or more of SEQ ID NO: 1-25, 31, 32.

55. A method for treating a disease that is associated with inflammation and/or fibrosis, comprising administering to a subject having the disease that exhibits inflammation and/or fibrosis a therapeutically effective amount of a formulation according to any one of claims 1 to 54.

56. Use of a formulation according to any one of claims 1 to 54 for the treatment of a disease associated with inflammation and/or fibrosis.

57. Use of a formulation according to any one of claims 1 to 54 for manufacture of a medicament for the treatment of a disease associated with inflammation and/or fibrosis.

58. The use of claim 55 or 56, wherein the disease comprises heart failure with preserved ejection fraction, myocardial infarction, muscular dystrophy, scleroderma, viral infection, and/or hypertrophic cardiomyopathy.

59. A method for manufacturing a formulation for oral delivery of a nucleic acid, comprising:
encapsulating a nucleic acid in an artificial lipid micelle by contacting the nucleic acid with a solution comprising cationic lipids, thereby generating an artificial lipid micelle comprising the nucleic acid;
coating the artificial lipid micelle comprising the nucleic acid with casein proteins by contacting the artificial lipid micelle comprising the nucleic acid with a solution comprising between 2 and 10% casein proteins, thereby generating a casein coated artificial lipid micelle comprising the nucleic acid;
exposing the casein coated artificial lipid micelle comprising the nucleic acid to a mixture of an acid and chitosan polymers, wherein the mixture of the acid and the chitosan polymers allows intercalation of the chitosan with the casein proteins and precipitation of casein-chitosan coated lipid micelles comprising the nucleic acid.

60. The method of claim 59, wherein the nucleic acid is contacted with the cationic lipids in a ratio of between about 10 to 30 ng of nucleic acid to 1 μL of lipid solution.

61. The method of claim 60, further comprising adding a liquid media to the nucleic acid and cationic lipid solution to a final volume of about 100 μL.

62. The method of claim 59, wherein the casein proteins are within a solution of 5% bovine casein solution and are added to the artificial lipid micelle comprising the nucleic acid at a volume ratio of 1:10.

63. The method of claim 59, wherein the mixture of the acid and the chitosan polymers comprises an acetic acid solution of between about 0.05 and 2% and a chitosan solution of between about 0.1% and 2%.

64. A method for treating or ameliorating a disease associated with inflammation and/or fibrosis, comprising:
administering to a subject in need thereof an oral formulation, comprising:
a nucleic acid;
a cationic lipid;
at least one casein protein; and
a chitosan.

65. The method of claim 64, wherein
the nucleic acid comprises a ribonucleic acid (RNA) and wherein the RNA is present in an amount ranging between about 0.0001 and 0.01% of the formulation by weight per volume;
wherein the at least one casein protein comprises at least an α-s1 casein subunit and wherein the at least one casein protein is present in an amount ranging between about 0.5 and 5% of the formulation by weight per volume; and
wherein the chitosan is present in an amount ranging between about 0.001 and 1% of the formulation by weight per volume.

66. The method of claim 65, wherein the formulation further comprises acetic acid, wherein the acetic acid is present in an amount ranging between about 0.01 and 1% of the formulation by weight per volume.

67. The method of claim 64, 65, or 66, wherein the cationic lipid is present in an amount ranging from about 0.1 to about 5 microliters for each microgram of nucleic acid.

68. A method according to any one of claims 64 to 67, wherein the nucleic acid comprises a non-coding RNA.

69. A method according to any one of claims 64 to 68, wherein the formulation further comprises an acid.

70. The method of claim 69, wherein the acid is present in an amount ranging between about 0.001 and 1% of the formulation by volume and where the acid is selected from acetic acid, citric acid, phosphoric acid and citric acid.

71. A method according to any one of claims 64 to 70, wherein the chitosan is low molecular weight chitosan.

72. The method of claim 71, wherein the low molecular weight chitosan ranges in mass from about 50 to about 190 kiloDaltons.

73. A method according to any one of claims 64 to 72, wherein the nucleic acid comprises a non-coding RNA, wherein the non-coding RNA is present in an amount ranging from between about 0.001 and about 0.005% of the formulation by weight per volume,
wherein the at least one casein protein comprises a mixture of an α-s1 casein subunit, an α-s2 casein subunit, a β casein subunit, and a κ casein subunit, wherein the casein subunits are present in an amount ranging between about 1 and 3% of the formulation by weight per volume; and
wherein the chitosan is present in an amount ranging between about 0.01 and 0.1% of the formulation by weight per volume.

74. The method of claim 73, wherein the non-coding RNA is present in an amount ranging from between about 0.002 and about 0.005% of the formulation by weight per volume,
wherein the mixture of casein subunits are present in an amount ranging between about 2 and 3% of the formulation by weight per volume;
wherein the chitosan is present in an amount ranging between about 0.05 and 0.1% of the formulation by weight per volume; and
wherein the cationic lipid is present in an amount ranging from about 1 to about 3 microliters for each microgram of nucleic acid.

75. The method of claim 74, wherein the non-coding RNA is present in an amount ranging from between about 0.0025 and about 0.004% of the formulation by volume,
wherein the mixture of casein subunits are present in an amount ranging between about 2.2 and 2.8% of the formulation by weight per volume;
wherein the chitosan is present in an amount ranging between about 0.06 and 0.09% of the formulation by weight per volume; and
wherein the cationic lipid is present in an amount ranging from about 1 to about 2 microliters for each microgram of nucleic acid.

76. The method of claim 75, wherein, upon administration to a subject, the non-coding RNA reduces expression of one or more of IL1-B, IL-6, TGF beta, NLRP3, p21, and IL-4.

77. The method of claim 75, wherein, upon administration to a subject, the non-coding RNA reduces systolic blood pressure of the subject.

78. The method of claim 75, wherein, upon administration to a subject, the non-coding RNA reduces diastolic blood pressure of the subject.

79. The method of claim 75, wherein, upon administration to a subject, the non-coding RNA enhances muscular endurance, muscular resistance to fatigue, muscular strength and/or muscle contractility of at least one muscle of the subject.

80. The method of claim 79, wherein, the muscle is skeletal muscle or cardiac muscle.

81. The method of claim 75, wherein, upon administration to a subject, the non-coding RNA reduces the expression of brain natriuretic peptide.

82. The method of claim 75, wherein, upon administration to a subject, the non-coding RNA reduces diastolic mitral inflow velocity to mitral annular tissue velocity (E/e′).

83. The method of claim 75, wherein, upon administration to a subject, the non-coding RNA enhances glucose tolerance within the subject.

84. The method of claim 75, wherein, upon administration to a subject, the non-coding RNA reduces obesity and/or subcutaneous adipose tissue per unit body mass of the subject.

85. The method of claim 75, wherein, upon administration to a subject having had a myocardial infarction, the non-coding RNA reduces infarct size after the myocardial infarction.

86. The method of claim 75, wherein, upon administration to a subject having had a myocardial infarction, the non-coding RNA reduces circulating cardiac troponin I concentration after the myocardial infarction.

87. A method according to any one of claims 64 to 86, wherein the disease is selected from heart failure with preserved ejection fraction, myocardial infarction, muscular dystrophy, scleroderma, viral infection, and hypertrophic cardiomyopathy.

88. A method according to any one of claims 64 to 87, wherein the nucleic acid comprises a sequence having at least 90% sequence identity to one or more of SEQ ID NO: 1-25, 31, 32.

89. A method according to any one of claims 64 to 88, wherein the nucleic acid consists essentially of a sequence having at least 90% sequence identity to one or more of SEQ ID NO: 1-25, 31, 32.
